SEQUENCE LISTING (13 sequences)

SEQ ID NO: 1 - protein, 119 aa, Homo sapiens [sequence omitted]

SEQ ID NO: 2 - protein, 160 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKP

SEQ ID NO: 3 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 4 - protein, 376 aa, Herpes simplex virus
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 5 - protein, 427 aa, Escherichia coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 6 - protein, 282 aa, Homo sapiens
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 7 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 8 - protein, 61 aa, Homo sapiens
MKRFLFLLLTISLLVMVQIQTGLSGQNDTSQTSSPSASSNISGGIFLFFVANAIIHLFCFS

SEQ ID NO: 9 - protein, 359 aa, Homo sapiens
MAEGTLWQILCVSSDAQPQTFEGVKGADPPTLPPGSFLPGPVLWWGSLARLQTEKSDEVSRKGNWWVTEMGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 10 - protein, 233 aa, Homo sapiens
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 11 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 12 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 13 - protein, 323 aa, Homo sapiens
MGILSFLPVLATESDWADCKSPQPWGHMLLWTAVLFLAPVAGTPAAPPKAVLKLEPQWINVLQEDSVTLTCRGTHSPESDSIPWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANSTDPVKAAQFEPPGRQMIAIRKRQPEETNNDYETADGGYMTLNPRAPTDDDKNIYLTLPPNDHVNSNN

(19) United States
(12) Patent Application Publication
(10) Pub. No.: US 2024/0091274 A1
(43) Pub. Date: Mar. 21, 2024

(54) TRANSPLANTED CELL PROTECTION VIA Fc SEQUESTRATION

(71) Applicant: The Regents of the University of California, Oakland, CA (US)

(72) Inventor: Tobias Deuse, Burlingame, CA (US)

(73) Assignee: The Regents of the University of California, Oakland, CA (US)

(21) Appl. No.: 17/768,217

(22) PCT Filed: Oct. 9, 2020
(86) PCT No.: PCT/US20/55120

§ 371 Date: Apr. 11, 2022

Related U.S. Application Data
(60) Provisional application No. 62/915,601, filed on Oct. 15, 2019.

(51) Int. Cl. A61K 35/545; A61K 39/00; C12N 5/074

(52) U.S. Cl. CPC A61K 35/545; A61K 39/4631; C12N 5/0696; C12N 2501/50; C12N 2506/02; C12N 2506/11; C12N 2506/45; C12N 2510/00

(57) ABSTRACT

The invention provides, for the first time, cells that comprise enhanced CD16, CD32, or CD64 expression to evade antibody-dependent cellular cytotoxicity (ADCC) or complement-dependent cytotoxicity (CDC). The cells may be pluripotent cells, including hypoimmune pluripotent cells (HIP) or ABO blood type O Rhesus Factor negative HIP cells (HIPO−), that further comprise the enhanced CD16, CD32, or CD64 expression. The invention encompasses cells derived from the pluripotent cells.

### I. CROSS REFERENCE TO RELATED APPLICATIONS

This application claims priority under 35 U.S.C. § 119(e) to U.S. Provisional Application No. 62/915,601, filed Oct. 15, 2019, which is incorporated herein by reference in its entirety.

### II. FIELD OF THE INVENTION

The invention relates to regenerative cell therapy. In some embodiments, the regenerative cell therapy comprises transplanting cell lines into patients in need thereof. In some embodiments, the cell lines comprise pluripotent cells with an elevated CD16, CD32, or CD64 expression. In other embodiments, they are hypoimmunogenic, have an O blood type, or are Rh factor negative. In other embodiments, the regenerative cell therapy reduces the propensity for the cell transplant recipient's immune system to reject allogeneic material. In some embodiments, the regenerative cell therapy is used in the treatment of injured organs and tissue. In some embodiments, the regenerative cell therapy of the invention utilizes chimeric antigen receptor (CAR) cells, endothelial cells, dopaminergic neurons, a pancreatic islet cells, cardiomyocytes, retinal pigment endothelium cells, or thyroid cells are used for treating diseases or rehabilitating damaged tissues.

### III. BACKGROUND OF THE INVENTION

Regenerative cell therapy is an important potential treatment for regenerating injured organs and tissues. With the low availability of organs for transplantation and the accompanying lengthy wait, the possibility of regenerating tissue by transplanting readily available cell lines into patients is understandably appealing. Regenerative cell therapy has shown promising initial results for rehabilitating damaged tissues after transplantation in animal models (e.g. after myocardial infarction). The propensity for the transplant recipient's immune system to reject allogeneic material, however, greatly reduces the potential efficacy of therapeutics and diminishes the possible positive effects surrounding such treatments.

Autologous induced pluripotent stem cells (iPSCs) theoretically constitute an unlimited cell source for patient-specific cell-based organ repair strategies. Their generation, however, poses technical and manufacturing challenges and is a lengthy process that conceptually prevents any acute treatment modalities. Allogeneic iPSC-based therapies or embryonic stem cell-based therapies are easier from a manufacturing standpoint and allow the generation of well-screened, standardized, high-quality cell products. Because of their allogeneic origin, however, such cell products would undergo rejection. With the reduction or elimination of the cells' antigenicity, universally-acceptable cell products could be produced. Because pluripotent stem cells can be differentiated into any cell type of the three germ layers, the potential application of stem cell therapy is wide-ranging. Differentiation can be performed ex vivo or in vivo by transplanting progenitor cells that continue to differentiate and mature in the organ environment of the implantation site. Ex vivo differentiation allows researchers or clinicians to closely monitor the procedure and ensures that the proper population of cells is generated prior to transplantation.

In most cases, however, undifferentiated pluripotent stem cells are avoided in clinical transplant therapies due to their propensity to form teratomas. Rather, such therapies tend to use differentiated cells (e.g. stem cell-derived cardiomyocytes transplanted into the myocardium of patients suffering from heart failure). Clinical applications of such pluripotent cells or tissues would benefit from a “safety feature” that controls the growth and survival of cells after their transplantation.

The art seeks stem cells capable of producing cells that are used to regenerate or replace diseased or deficient cells. Pluripotent stem cells (PSCs) may be used because they rapidly propagate and differentiate into many possible cell types. The family of PSCs includes several members generated via different techniques and possessing distinct immunogenic features. Patient compatibility with engineered cells or tissues derived from PSCs determines the risk of immune rejection and the requirement for immunosuppression.

Embryonic stem cells (ESCs) isolated from the inner cell mass of blastocysts exhibit the histocompatibility antigens that are mismatches with recipients. This immunological barrier cannot be solved by human leukocyte antigen (HLA)-typed banks of ESCs because even HLA-matched PSC grafts undergo rejection because of mismatches in non-HLA molecules that function as minor antigens. This is also true for allogeneic induced pluripotent stem cells (iPSCs).

Hypoimmunogenic pluripotent (HIP) cells and cell products have gene knockouts or transgenes to protect them from the cellular components of the immune system that include T cells, NK cells, and macrophages. They may also be ABO blood group type O and Rh negative (HIPO−).

The two most relevant killing mechanisms involving antibodies are antibody-dependent cellular cytotoxicity (ADCC) by NK cells, macrophages, B-cells or granulocytes and complement-dependent cytotoxicity (CDC) via activation of the complement cascade. All of those killing mechanisms utilize antibodies that bind to a target cell and activate the effector immune cells or complement (FIG. 1A). IgG antibodies can be potent mediations of both ADCC and CDC. IgG antibodies have two variable Fab regions which bind to specific epitopes. The crystalizable Fc region sticks out and serves for the binding of NK cells, B-cells, macrophages, granulocytes, or complement.

Receptors that recognize the Fc portion of IgG are divided into four different classes: FcγRI (CD64), FcγRII (CD32), FcγRIII (CD16), and FcγRIV. Whereas FcγRI displays high affinity for the antibody constant region and restricted isotype specificity, FcγRII and FcγRIII have low affinity for the Fc region of IgG but a broader isotype binding pattern, and FcγRIV is a recently identified receptor with intermediate affinity and restricted subclass specificity. Physiologically, FcγRI functions during early immune responses, while FcγRII and RIII recognize IgG as aggregates surrounding multivalent antigens during late immune responses.

If an antibody binds to an unprotected cell via its Fab regions, the FC can be bound by NK cells (mostly via their CD16 receptor), macrophages (mostly via CD16, CD32, or CD64), B-cells (mostly via CD32), or granulocytes (mostly via CD16, CD32, or CD64) and mediate ADCC. Complement can also bind to Fc and activate its cascade and form the membrane attack complex (MAC) for CDC killing.

In humans, CD16 comes as CD16A and CD16B, which have 96% sequence similarity in the extracellular immunoglobulin binding regions (aka. FCGR3A and FCGR3B). For FCGR3A, there are several isoforms and there is no clear principal isoform.

Pluripotent cells currently have no protection against ADCC or CDC.

### IV. SUMMARY OF THE INVENTION

The invention provides, for the first time, cells that comprise enhanced CD16, CD32, or CD64 expression to evade antibody-dependent cellular cytotoxicity (ADCC) or complement-dependent cytotoxicity (CDC). Given the lack of downstream activation or inhibitory motifs in target cells usually not expressing Fcγ receptors, the high Fc affinity of CD64 was for protective Fc sequestration. The overexpression of either one of the Fcγ receptors I-IV, or a combination of those, should also show some or even enhanced efficacy.

The invention provides that CD16, CD32, or CD64 overexpression sequesters the Fc portion of local antibodies and thus inhibits ADCC and CDC. (FIG. 1B). The cells may be pluripotent cells, including hypoimmune pluripotent cells (HIP), ABO blood type O Rhesus Factor negative HIP cells (HIPO−) induced pluripotent stem cells (iPSC), iPSCs that are O−, embryonic stem cells (ESC), or ESCs that are O−, any of which further comprise the enhanced CD64 expression.

CD64 is constitutively found only on macrophages and monocytes and is usually not expressed on tissue cells. It is more commonly known as Fc-gamma receptor 1 (FcγRI) and binds IgG Fc regions with high affinity. CD64 overexpression on target cells sequesters the IgG Fc and binds it to the target cell, which has no intracellular motifs for cellular activation. Even if the free Fab regions would bind neighboring target cells, the occupation of the Fc prevents any ADCC or CDC

Thus, the invention provides a modified pluripotent cell, wherein said modified cell has an elevated level of CD16, CD32, or CD64 protein expression when compared to a parental version of said modified pluripotent cell, wherein said elevated protein expression causes said modified pluripotent cell to be less susceptible to antibody dependent cellular cytoxicity (ADCC) or complement-dependent cytotoxicity (CDC). In some aspects, the CD64 protein has at least a 90% sequence identity to SEQ ID NO:7. In other aspects, the CD64 protein has the sequence of SEQ ID NO:7. In some aspects, the CD16 protein has at least a 90% sequence identity to SEQ ID NOS:9 or 10. In other aspects, the CD16 protein has the sequence of SEQ ID NOS:9 or 10. In some aspects, the CD32 protein has at least a 90% sequence identity to SEQ ID NOS:11, 12, or 13. In other aspects, the CD32 protein has the sequence of SEQ ID NOS:11, 12, or 13.

In some aspects of the invention, the modified cell is derived from a human hypo-immunogenic pluripotent (HIP) cell, a human hypo-immunogenic pluripotent ABO blood group O Rhesus Factor negative (HIPO−) cell, a human induced pluripotent stem cell (iPSC), or a human embryonic stem cell (ESC).

In other aspects of the invention, the modified cell is from a species that is selected from the group consisting of a human, monkey, cow, pig, chicken, turkey, horse, sheep, goat, donkey, mule, duck, goose, buffalo, camel, yak, llama, alpaca, mouse, rat, dog, cat, hamster, and guinea pig.

The invention provides a modified pluripotent cell as described herein, further comprising a suicide gene that is activated by a trigger that causes the modified cell to die. In some aspects, the suicide gene is a herpes simplex virus thymidine kinase gene (HSV-tk) and the trigger is ganciclovir. In other aspects, the HSV-tk gene encodes a protein comprising at least a 90% sequence identity to SEQ ID NO:4. In a preferred aspect, the HSV-tk gene encodes a protein comprising the sequence of SEQ ID NO:4. In other aspects of the invention, the suicide gene is an Escherichia coli cytosine deaminase gene (EC-CD) and the trigger is 5-fluorocytosine (5-FC). In preferred aspects, the EC-CD gene encodes a protein comprising at least a 90% sequence identity to SEQ ID NO:5. In more preferred aspects, the EC-CD gene encodes a protein comprising the sequence of SEQ ID NO:5.

In some aspects of the invention, the suicide gene encodes an inducible Caspase protein and the trigger is a chemical inducer of dimerization (CID). In preferred aspects, the gene encodes an inducible Caspase protein comprising at least a 90% sequence identity to SEQ ID NO:6. In more preferred aspects, the gene encodes an inducible Caspase protein comprising the sequence of SEQ ID NO:6. In another preferred aspect, the CID is AP1903.

The invention provides a cell derived from the modified pluripotent cells described herein, wherein the derivative cell is selected from the group consisting of a chimeric antigen receptor (CAR) cell, an endothelial cell, a dopaminergic neuron, a pancreatic islet cell, a cardiomyocyte, a retinal pigment endothelium cell, and a thyroid cell. In some aspects, the CAR cell is a CAR-T cell.

The invention provides a method, comprising transplanting a cell derived from the modified pluripotent cells disclosed herein into a subject, wherein the subject is a human, monkey, cow, pig, chicken, turkey, horse, sheep, goat, donkey, mule, duck, goose, buffalo, camel, yak, llama, alpaca, mouse, rat, dog, cat, hamster, guinea pig. In some aspects, the cell derived from the modified pluripotent cell is selected from the group consisting of a chimeric antigen receptor (CAR) cell, an endothelial cell, a dopaminergic neuron, a pancreatic islet cell, a cardiomyocyte, a retinal pigment endothelium cell, and a thyroid cell.

The invention provides a method of treating a disease, comprising administering a cell derived from the modified pluripotent cell as disclosed herein. In some aspects, the derivative cell is selected from the group consisting of a chimeric antigen receptor (CAR) cell, an endothelial cell, a dopaminergic neuron, a pancreatic islet cell, a cardiomyocyte, a retinal pigment endothelium cell, and a thyroid cell. In other aspects, the disease is selected from the group consisting of Type I Diabetes, a cardiac disease, a neurological disease, a cancer, an ocular disease, a vascular disease, and a thyroid disease.

The invention provides a method for generating the modified pluripotent cells as disclosed herein, comprising increasing the expression of CD16, CD32, or CD64 in the parental non-modified version of the pluripotent cell. In some aspects, the modified cell has a human, monkey, cow, pig, chicken, turkey, horse, sheep, goat, donkey, mule, duck, goose, buffalo, camel, yak, llama, alpaca, mouse, rat, dog, cat, hamster, or guinea pig origin. In some aspects, the modified pluripotent cell is derived from a HIP cell, a HIPO− cell, an iPSC cell, or an ESC cell.

In some aspects of the invention, the increased CD16, CD32, or CD64 expression results from introducing at least one copy of a human CD16, CD32, or CD64 gene under the control of a promoter into the parental version of the modified pluripotent cell. In preferred aspects, the promoter is a constitutive promoter.

The invention provides a pharmaceutical composition for treating a disease, comprising a cell derived from the modified pluripotent cell of any one of claims 1-19 and a pharmaceutically-acceptable carrier. In some aspects, the derivative cell is selected from the group consisting of a chimeric antigen receptor (CAR) cell, an endothelial cell, a dopaminergic neuron, a pancreatic islet cell, a cardiomyocyte, a retinal pigment endothelium cell, and a thyroid cell. In other aspects, the disease is selected from the group consisting of Type I Diabetes, a cardiac disease, a neurological disease, a cancer, an ocular disease, a vascular disease, and a thyroid disease.

The invention provides a medicament for treating a disease, comprising a cell derived from the modified pluripotent cells disclosed herein. In some aspects, the derivative cell is selected from the group consisting of a chimeric antigen receptor (CAR) cell, an endothelial cell, a dopaminergic neuron, a pancreatic islet cell, a cardiomyocyte, a retinal pigment endothelium cell, and a thyroid cell. In other aspects, the disease is selected from the group consisting of Type I Diabetes, a cardiac disease, a neurological disease, a cancer, an ocular disease, a vascular disease, and a thyroid disease.

The invention provides a modified cell, comprising an elevated level of CD16, CD32, or CD64 protein expression when compared to a parental version of the modified cell, wherein the elevated protein expression causes the modified cell to be less susceptible to antibody dependent cellular cytoxicity (ADCC) or complement-dependent cytotoxicity (CDC). In some aspects, the cell is selected from the group consisting of a chimeric antigen receptor (CAR) cell, an endothelial cell, a dopaminergic neuron, a pancreatic islet cell, a cardiomyocyte, a retinal pigment endothelium cell, and a thyroid cell.

### V. BRIEF DESCRIPTION OF THE DRAWINGS

FIG. 1 is a schematic diagram of ADCC and CDC.

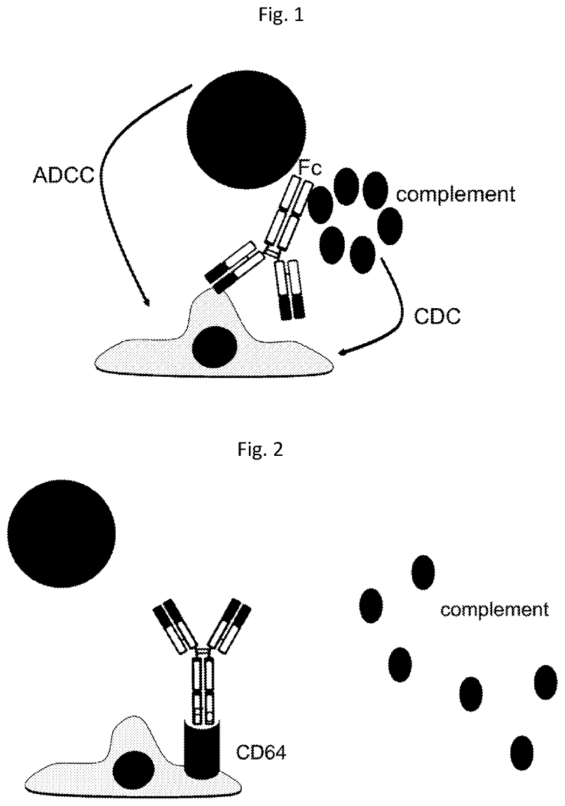

FIG. 2 is a schematic diagram of the invention. Cells expressing CD64 sequester antibody Fc domains and prevent ADCC or CDC.

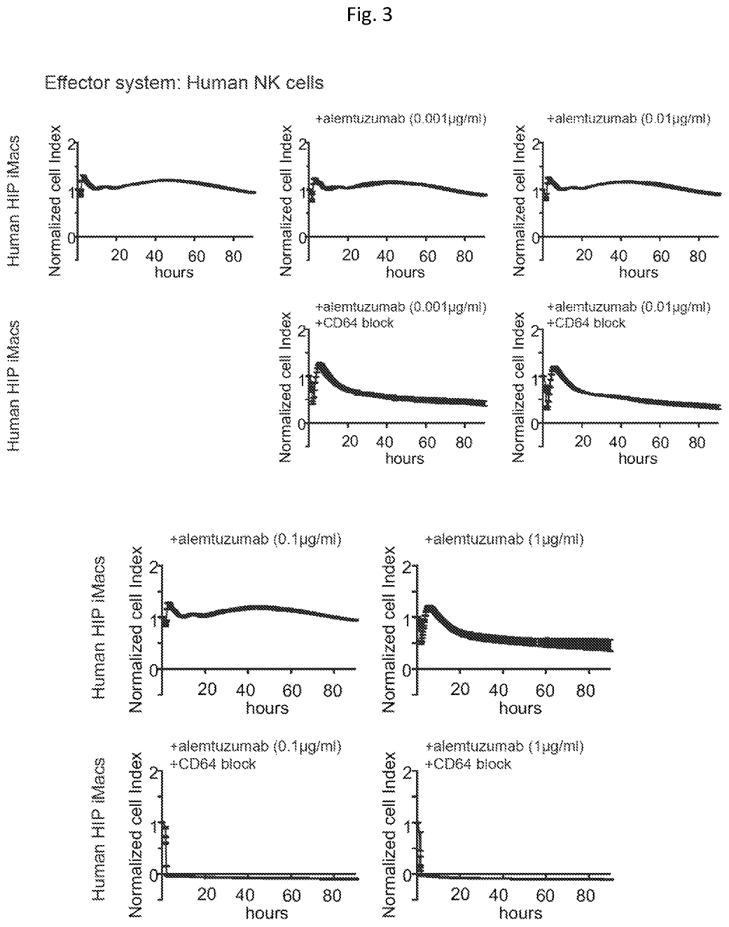

FIG. 3 shows that CD52+ human macrophages were protected from anti-CD52 antibody (alemtuzumab)-mediated ADCC by their constitutive expression of CD64. Anti-CD64 antibodies ablated the protection. This was most apparent at the lower anti-CD52 antibody concentrations.

FIG. 4 shows CD52+ human HIP iPSC-derived endothelial cells were protected by CD64 from NK cell ADCC resulting from anti-CD52 antibodies (alemtuzumab). Anti-CD64 antibodies ablated the protection. The presence of a CD64 transgene significantly rescued the endothelial cells at all but the highest level of anti-CD52 antibody.

FIG. 5 shows that CD52+ macrophages are protected from anti-CD52 antibody (alemtuzumab)-mediated CDC by their constitutive expression of CD64. Anti-CD64 antibodies ablated the protection. This was most apparent at the lower anti-CD52 antibody concentrations.

FIGS. 6A-6C show mouse HIP iECs engineered to express CD52 (FIG. 6A), CD52 and CD64 (FIG. 6B), and CD64 (FIG. 6C).

FIGS. 7A and 7B show that mouse HIP iECs were unable to bind alemtuzumab Fc (FIG. 7A), but mouse HIP iECs (CD64) bind alemtuzumab Fc in a concentration-dependent manner (FIG. 7B).

FIG. 8 shows that CD52+ mouse HIP iECs were susceptible to anti-CD52 antibody (alemtuzumab)-mediated syngeneic NK cell ADCC in a concentration-dependent manner (upper row), but they were protected from NK cell killing if they co-expressed CD64 (lower row).

FIG. 9 shows that CD52+ mouse HIP iECs were susceptible to anti-CD52 antibody (alemtuzumab)-mediated syngeneic macrophage ADCC in a concentration-dependent manner (upper row), but they were protected from macrophage killing if they co-expressed CD64 (lower row).

FIG. 10 shows that CD52+ mouse HIP iECs were susceptible to anti-CD52 antibody (alemtuzumab)-mediated syngeneic PMN ADCC in a concentration-dependent manner (upper row), but they were protected from PMN killing if they co-expressed CD64 (lower row).

FIG. 11 shows that CD52+ mouse B6 HIP iECs were susceptible to anti-CD52 antibody (alemtuzumab)-mediated ADCC by allogeneic BALB/c NK cells in a concentration-dependent manner (upper row), but they were protected from BALB/c NK cell killing if they co-expressed CD64 (lower row).

FIG. 12 shows that CD52+ mouse B6 HIP iECs were susceptible to anti-CD52 antibody (alemtuzumab)-mediated ADCC by allogeneic BALB/c macrophages in a concentration-dependent manner (upper row), but they were protected from BALB/c macrophage killing if they co-expressed CD64 (lower row).

FIG. 13 shows that CD52+ mouse B6 HIP iECs were susceptible to anti-CD52 antibody (alemtuzumab)-mediated ADCC by allogeneic PMNs in a concentration-dependent manner (upper row), but they were protected from BALB/c PMN killing if they co-expressed CD64 (lower row).

FIG. 14 shows that CD52+ mouse B6 HIP iECs were susceptible to anti-CD52 antibody (alemtuzumab)-mediated CDC in a concentration-dependent manner (upper row), and since they did not express CD64, an anti-CD64 antibody did not affect this killing (lower row).

FIG. 15 shows that CD52+CD64+ mouse B6 HIP iECs were protected from anti-CD52 antibody (alemtuzumab)-mediated CDC across all concentrations tested, and an anti-CD64 antibody ablated this protection (lower row).

FIGS. 16A-16C show human HIP iECs engineered to express CD52 (FIG. 16A), CD52 and CD64 (FIG. 16B), and CD64 (FIG. 16C).

FIGS. 17A and 17B show that human HIP iECs were unable to bind alemtuzumab Fc (FIG. 17A), but human HIP iECs (CD64) bound alemtuzumab Fc in a concentration-dependent manner (FIG. 17B).

FIG. 18 shows that human macrophages, which constitutively express CD64, bound alemtuzumab Fe in a concentration-dependent manner.

FIG. 19 shows that CD52+ human HIP iECs were susceptible to anti-CD52 antibody (alemtuzumab)-mediated NK cell ADCC in a concentration-dependent manner (upper row), but they were mostly protected from allogeneic NK cell killing if they co-expressed CD64 (lower row). Only the highest anti-CD52 antibody concentration caused some cytotoxicity.

FIG. 20 shows that CD52+ human HIP iECs were susceptible to anti-CD52 antibody (alemtuzumab)-mediated allogeneic macrophage ADCC in a concentration-dependent manner (upper row), but they were mostly protected from macrophage killing if they co-expressed CD64 (lower row). Only the highest anti-CD52 antibody concentration caused some cytotoxicity.

FIG. 21 shows that CD52+ human HIP iECs were susceptible to anti-CD52 antibody (alemtuzumab)-mediated CDC in a concentration-dependent manner (upper row), and since they did not express CD64, an anti-CD64 antibody did not affect this killing (lower row).

FIG. 22 shows that CD52+CD64+ human HIP iECs were mostly protected from anti-CD52 antibody (alemtuzumab)-mediated CDC. Only the highest anti-CD52 antibody concentration caused some cytotoxicity. An anti-CD64 antibody ablated this protection (lower row).

FIGS. 23A and B show human (FIG. 23A) and mouse (FIG. 23B) thyroid epithelial cells (epiCs) engineered to express CD64. Human epiCs were unable to bind alemtuzumab Fc, but human epiCs (CD64) bound alemtuzumab Fc in a concentration-dependent manner (FIG. 23C). Mouse epiCs were unable to bind alemtuzumab Fc, but mouse epiCs (CD64) bound alemtuzumab Fc in a concentration-dependent manner (FIG. 23D).

FIG. 24 shows that human epiCs were unable to bind anti-TPO Fc, but human epiCs (CD64) bound anti-TPO Fc in a concentration-dependent manner (FIG. 24A). Mouse epiCs were unable to bind anti-TPO Fc, but mouse epiCs (CD64) bound anti-TPO Fc in a concentration-dependent manner (FIG. 24B).

FIG. 25 shows that C57BL/6 thyroid epiCs were susceptible to anti-TPO-mediated killing when incubated with syngeneic macrophages as effector cells for ADCC.

FIG. 26 shows that C57BL/6 thyroid epiCs expressing CD64 were not susceptible to anti-TPO-mediated killing when incubated with syngeneic macrophages as effector cells for ADCC. Across a wide range of anti-TPO concentrations, the target cells were protected from anti-TPO ADCC.

FIGS. 27A and 27B show a clinically relevant in vivo model for antibody-mediated rejections. Mouse HIP iECs carrying CD52 were killed when syngeneic recipients were treated with alemtuzumab (FIG. 27A). Mouse HIP iECs carrying CD52 and expressing CD64, however, were fully protected from antibody-mediated rejection.

### VI. DETAILED DESCRIPTION OF THE INVENTION

### A. Introduction

The invention provides, for the first time, cells that comprise enhanced CD16, CD32, or CD64 expression to evade antibody-dependent cellular cytotoxicity (ADCC) or complement-dependent cytotoxicity (CDC). The cells may be pluripotent cells, including hypoimmune pluripotent cells (HIP) or ABO blood type O Rhesus Factor negative HIP cells (HIPO−), that further comprise the enhanced CD64 expression.

One specific indication for this technology can be the cellular treatment of autoimmune diseases. Antibodies against autologous epitopes cause beta cell death in type 1 diabetes and thyroid cells in autoimmune thyroiditis. Antibodies may bind to HLA class II molecules. Additionally, HLA-independent antibodies have been described. Should a cell that is used for regenerative therapy have the same epitope, it will also be bound by these antibodies and be killed. The CD64 sequestration of the invention prevents ADCC or CDC.

In some embodiments of the invention, HypoImmunogenic Pluripotent (“HIP”) cells are modified to enhance CD16, CD32, or CD64 expression (HIP/CD16, HIP/CD32, or HIP/CD64 cells). HIP cells avoid host immune responses due to several genetic manipulations as outlined herein. The cells lack major immune antigens that trigger immune responses and are engineered to avoid phagocytosis and NK cell killing. In some embodiments, the HIP cells are made by eliminating the activity of both alleles of a B2M gene in an induced pluripotent stem cell (iPSC); eliminating the activity of both alleles of a CIITA gene in the iPSC; and increasing the expression of CD47 in the iPSC. HIP cells are described in detail in WO2018132783, incorporated by reference herein in its entirety.

In other embodiments of the invention, HypoImmunogenic Pluripotent Blood group O Rh—(“HIPO−”) cells are modified to enhance CD16, CD32, or CD64 expression (HIPO−/CD16 cells, HIPO−/CD32, or HIPO−/CD64 cells). HIPO− cells avoid host immune responses due to several genetic or enzymatic manipulations as outlined herein. The cells lack major blood group and immune antigens that trigger immune responses and are engineered to avoid rejection, phagocytosis, or killing. This allows the derivation of “off-the-shelf” cell products for generating specific tissues and organs. The benefit of being able to use human allogeneic HIPO− cells and their derivatives in human patients provides significant benefits, including the ability to avoid long-term adjunct immunosuppressive therapy and drug use generally seen in allogeneic transplantations. They also provide significant cost savings as cell therapies can be used without requiring individual treatments for each patient. HIPO−/CD16, HIPO−/CD32, or HIPO−/CD64 cells may serve as a universal cell source for the generation of universally-acceptable derivatives. HIPO− cells are described in detail in U.S. Prov. Appl. Nos. 62/846,399 and 62,855,499 each of which are incorporated by reference herein in their entirety.

### B. Definitions

The term “pluripotent cells” refers to cells that can self-renew and proliferate while remaining in an undifferentiated state and that can, under the proper conditions, be induced to differentiate into specialized cell types. The term “pluripotent cells,” as used herein, encompass embryonic stem cells and other types of stem cells, including fetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J. Med. 350:13. (2004), incorporated by reference herein in its entirety.)

“Pluripotent stem cells” as used herein have the potential to differentiate into any of the three germ layers: endoderm (e.g. the stomach linking, gastrointestinal tract, lungs, etc.), mesoderm (e.g. muscle, bone, blood, urogenital tissue, etc.) or ectoderm (e.g. epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells, and “miPSCs” are murine induced pluripotent stem cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. As described herein, cells do not need to pass through pluripotency to be reprogrammed into endodermal progenitor cells and/or hepatocytes.

As used herein, “multipotent” or “multipotent cell” refers to a cell type that can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “totipotent” means the ability of a cell to form an entire organism. For example, in mammals, only the zygote and the first cleavage stage blastomeres are totipotent.

As used herein, “non-pluripotent cells” refer to mammalian cells that are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells. The starting cells employed for generating the induced multipotent cells, the endodermal progenitor cells, and the hepatocytes can be non-pluripotent cells.

Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells.

Cells can be from, for example, human or non-human mammals. Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal. In some embodiments, a cell is from a neonatal human, an adult human, or non-human mammal.

As used herein, the terms “subject” or “patient” refers to any animal, such as a domesticated animal, a zoo animal, or a human. The “subject” or “patient” can be a mammal like a dog, cat, bird, livestock, or a human. Specific examples of “subjects” and “patients” include, but are not limited to, individuals (particularly human) with a disease or disorder related to the liver, heart, lung, kidney, pancreas, brain, neural tissue, blood, bone, bone marrow, and the like.

Mammalian cells can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

By “hypo-immunogenic pluripotent” cell or “HIP” cell herein is meant a pluripotent cell that retains its pluripotent characteristics and yet gives rise to a reduced immunological rejection response when transferred into an allogeneic host. In preferred embodiments, HIP cells do not give rise to an immune response. Thus, “hypo-immunogenic” refers to a significantly reduced or eliminated immune response when compared to the immune response of a parental (i.e. “wt”) cell prior to immunoengineering as outlined herein. In many cases, the HIP cells are immunologically silent and yet retain pluripotent capabilities. Assays for HIP characteristics are outlined below.

By “HIP/CD16”, “HIP/CD32”, or “HIP/CD64” cell herein is meant a HIP cell that has enhanced CD16, CD32, or CD64 expression, respectively.

By “hypo-immunogenic pluripotent cell 0-” “hypo-immunogenic pluripotent ORh−” cell or “HIPO−” cell herein is meant a HIP cell that is also ABO blood group O and Rhesus Factor Rh−. HIPO− cells may have been generated from O− cells, enzymatically modified to be O−, or genetically engineered to be O−.

By “HIPO−/CD16”, “HIPO−/CD32”, or “HIPO−/CD64” cell herein is meant a HIPO-cell that has enhanced CD16, CD32, or CD64 expression.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with 3-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

By “gene knock out” herein is meant a process that renders a particular gene inactive in the host cell in which it resides, resulting either in no protein of interest being produced or an inactive form. As will be appreciated by those in the art and further described below, this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

By “gene knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

“3-2 microglobulin” or “02M” or “B2M” protein refers to the human 02M protein that has the amino acid and nucleic acid sequences shown below; the human gene has accession number NC_000015.10:44711487-44718159.

“CD47 protein” protein refers to the human CD47 protein that has the amino acid and nucleic acid sequences shown below; the human gene has accession number NC_000016.10:10866208-10941562.

“CIITA protein” protein refers to the human CIITA protein that has the amino acid and nucleic acid sequences shown below; the human gene has accession number NC_000003.12:108043094-108094200.

By “wild type” in the context of a cell means a cell found in nature. However, in the context of a pluripotent stem cell, as used herein, it also means an iPSC that may contain nucleic acid changes resulting in pluripotency but did not undergo the gene editing procedures of the invention to achieve hypo-immunogenicity.

By “syngeneic” herein refers to the genetic similarity or identity of a host organism and a cellular transplant where there is immunological compatibility; e.g. no immune response is generated.

By “allogeneic” herein refers to the genetic dissimilarity of a host organism and a cellular transplant where an immune response is generated.

By “B2M−/−” herein is meant that a diploid cell has had the B2M gene inactivated in both chromosomes. As described herein, this can be done in a variety of ways.

By “CIITA −/−” herein is meant that a diploid cell has had the CIITA gene inactivated in both chromosomes. As described herein, this can be done in a variety of ways.

By “CD47 tg” (standing for “transgene”) or “CD47+”) herein is meant that the host cell expresses CD47, in some cases by having at least one additional copy of the CD47 gene.

An “Oct polypeptide” refers to any of the naturally-occurring members of Octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include Oct-1, Oct-2, Oct-3/4, Oct-6, Oct-7, Oct-8, Oct-9, and Oct-11. Oct3/4 (referred to herein as “Oct4”) contains the POU domain, a 150 amino acid sequence conserved among Pit-1, Oct-1, Oct-2, and uric-86. (See, Ryan, A. K. & Rosenfeld, M. G., Genes Dev. 11:1207-1225 (1997), incorporated herein by reference in its entirety.) In some embodiments, variants have at least 85%, 90%, or 95% amino acid sequence identity across their whole sequence compared to a naturally occurring Oct polypeptide family member such as to those listed above or such as listed in Genbank accession number NP-002692.2 (human Oct4) or NP-038661.1 (mouse Oct4). Oct polypeptides (e.g., Oct3/4 or Oct 4) can be from human, mouse, rat, bovine, porcine, or other animals. Generally, the same species of protein will be used with the species of cells being manipulated. The Oct polypeptide(s) can be a pluripotency factor that can help induce multipotency in non-pluripotent cells.

A “Klf polypeptide” refers to any of the naturally-occurring members of the family of Krippel-like factors (Klfs), zinc-finger proteins that contain amino acid sequences similar to those of the Drosophila embryonic pattern regulator Kruppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. (See, Dang, D. T., Pevsner, J. & Yang, V. W., Cell Biol. 32:1103-1121 (2000), incorporated by reference herein in its entirety.) Exemplary Klf family members include, Klf1, Klf2, Klf3, Klf-4, Klf5, Klf6, Klf7, Klf8, Klf9, Klf10, Klf11, Klf12, Klf13, Klf14, Kif15, Klf16, and Klf17. Klf2 and Klf-4 were found to be factors capable of generating iPS cells in mice, and related genes Klf1 and Klf5 did as well, although with reduced efficiency. (See, Nakagawa, et al., Nature Biotechnology 26:101-106 (2007), incorporated by reference herein in its entirety.) In some embodiments, variants have at least 85%, 90%, or 95% amino acid sequence identity across their whole sequence compared to a naturally occurring Klf polypeptide family member such as to those listed above or such as listed in Genbank accession number CAX16088 (mouse Klf4) or CAX14962 (human Klf4). Klf polypeptides (e.g., Klf1, Klf4, and Klf5) can be from human, mouse, rat, bovine, porcine, or other animals. Generally, the same species of protein will be used with the species of cells being manipulated. The Klf polypeptide(s) can be a pluripotency factor. The expression of the Klf4 gene or polypeptide can help induce multipotency in a starting cell or a population of starting cells.

A “Myc polypeptide” refers to any of the naturally-occurring members of the Myc family. (See, e.g., Adhikary, S. & Eilers, M., Nat. Rev. Mol. Cell Biol. 6:635-645 (2005), incorporated by reference herein in its entirety.) It also includes variants that maintain similar transcription factor activity when compared to the closest related naturally occurring family member (i.e. within at least 50%, 80%, or 90% activity). It further includes polypeptides comprising at least the DNA-binding domain of a naturally occurring family member, and can further comprise a transcriptional activation domain. Exemplary Myc polypeptides include, e.g., c-Myc, N-Myc and L-Myc. In some embodiments, variants have at least 85%, 90%, or 95% amino acid sequence identity across their whole sequence compared to a naturally occurring Myc polypeptide family member, such as to those listed above or such as listed in Genbank accession number CAA25015 (human Myc). Myc polypeptides (e.g., c-Myc) can be from human, mouse, rat, bovine, porcine, or other animals. Generally, the same species of protein will be used with the species of cells being manipulated. The Myc polypeptide(s) can be a pluripotency factor.

A “Sox polypeptide” refers to any of the naturally-occurring members of the SRY-related HMG-box (Sox) transcription factors, characterized by the presence of the high-mobility group (HMG) domain, or variants thereof that maintain similar transcription factor activity when compared to the closest related naturally occurring family member (i.e. within at least 50%, 80%, or 90% activity). It also includes polypeptides comprising at least the DNA-binding domain of the naturally occurring family member, and can further comprise a transcriptional activation domain. (See, e.g., Dang, D. T. et al., Int. J Biochem. Cell Biol. 32:1103-1121 (2000), incorporated by reference herein in its entirety.) Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. Sox1 has been shown to yield iPS cells with a similar efficiency as Sox2, and genes Sox3, Sox15, and Sox18 have also been shown to generate iPS cells, although with somewhat less efficiency than Sox2. (See, Nakagawa, et al., Nature Biotechnology 26:101-106 (2007), incorporated by reference herein in its entirety.) In some embodiments, variants have at least 85%, 90%, or 95% amino acid sequence identity across their whole sequence compared to a naturally occurring Sox polypeptide family member such as to those listed above or such as listed in Genbank accession number CAA83435 (human Sox2). Sox polypeptides (e.g., Sox1, Sox2, Sox3, Sox15, or Sox18) can be from human, mouse, rat, bovine, porcine, or other animals.

Generally, the same species of protein will be used with the species of cells being manipulated. The Sox polypeptide(s) can be a pluripotency factor. As discussed herein, SOX2 proteins find particular use in the generation of iPSCs.

By “differentiated hypo-immunogenic pluripotent cells” or “differentiated HIP cells” or “dHIP cells” herein is meant iPS cells that have been engineered to possess hypoimmunogenicity (e.g. by the knock out of B2M and CIITA and the knock in of CD47) and then are differentiated into a cell type for ultimate transplantation into subjects. Thus, for example HIP cells can be differentiated into hepatocytes (“dHIP hepatocytes”), into beta-like pancreatic cells or islet organoids (“dHIP beta cells”), into endothelial cells (“dHIP endothelial cells”), etc. Paralell definitions apply to “differentiated HIP/CD64” and differentiated HIPO−/CD64 cells.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat′l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

“Inhibitors,” “activators,” and “modulators” affect a function or expression of a biologically-relevant molecule. The term “modulator” includes both inhibitors and activators. They may be identified using in vitro and in vivo assays for expression or activity of a target molecule.

“Inhibitors” are agents that, e.g., inhibit expression or bind to target molecules or proteins. They may partially or totally block stimulation or have protease inhibitor activity. They may reduce, decrease, prevent, or delay activation, including inactivation, desensitizion, or down regulation of the activity of the described target protein. Modulators may be antagonists of the target molecule or protein.

“Activators” are agents that, e.g., induce or activate the function or expression of a target molecule or protein. They may bind to, stimulate, increase, open, activate, or facilitate the target molecule activity. Activators may be agonists of the target molecule or protein.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least an 50, 55, 60, 65, 70, 75, 85, 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers (1995), incorporated by reference herein in its entirety.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 Mm sodium phosphate buffer at Ph 6.5 with 750 Mm sodium chloride, 75 Mm sodium citrate at 42° C.; or (3) overnight hybridization in a solution that employs 50% formamide, 5×SSC (0.75 M NaCl, 0.075 M sodium citrate), 50 Mm sodium phosphate (Ph 6.8), 0.1% sodium pyrophosphate, 5×Denhardt's solution, sonicated salmon sperm DNA (50 μl/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein the term “modification” refers to an alteration that physically differentiates the modified molecule from the parent molecule. In one embodiment, an amino acid change in a CD47, HSVtk, EC-CD, or iCasp9 variant polypeptide prepared according to the methods described herein differentiates it from the corresponding parent that has not been modified according to the methods described herein, such as wild-type proteins, a naturally occurring mutant proteins or another engineered protein that does not include the modifications of such variant polypeptide. In another embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide.

In one embodiment, a variant polypeptide includes one or more substitutions, insertions, or deletions relative to a corresponding native or parent sequence. In certain embodiments, a variant polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41 to 50, or 51 or more modifications.

By “episomal vector” herein is meant a genetic vector that can exist and replicate autonomously in the cytoplasm of a cell; e.g. it is not integrated into the genomic DNA of the host cell. A number of episomal vectors are known in the art and described below.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knock out is effected at the genomic DNA level, such that the cells' offspring also carry the knock out permanently.

By “knock in” in the context of a gene means that the host cell harboring the knock in has more functional protein active in the cell. As outlined herein, a knock in can be done in a variety of ways, usually by the introduction of at least one copy of a transgene (tg) encoding the protein into the cell, although this can also be done by replacing regulatory components as well, for example by adding a constitutive promoter to the endogeneous gene. In general, knock in technologies result in the integration of the extra copy of the transgene into the host cell.

### VII. CELLS OF THE INVENTION

The invention provides compositions and methodologies for generating pluripotent cells with enhanced CD16, CD32, OR CD64 expression. In some aspects of the invention, the cells will be derived from induced pluripotent stem cells (IPSC), O− induced pluripotent stem cells (iPSCO−), embryonic stem cells (ESC), O− embryonic stem cells (ESCO−), hypoimmunogenic pluripotent (HIP) cells, hypoimmunogenic pluripotent O−(HIPO−) cells, or cells derived or differentiated therefrom.

### A. Methodologies for Genetic Alterations

The invention includes methods of modifying nucleic acid sequences within cells or in cell-free conditions to generate both cells with enhanced CD16, CD32, or CD64 expression. Exemplary technologies include homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

As will be appreciated by those in the art, a number of different techniques can be used to engineer the pluripotent cells of the invention, as well as the engineering of the iPSCs to become hypo-immunogenic as outlined herein.

In general, these techniques can be used individually or in combination. For example, in the generation of the HIP cells, CRISPR may be used to reduce the expression of active B2M and/or CIITA protein in the engineered cells, with viral techniques (e.g. lentivirus) to knock in the CD47 functionality. Also, as will be appreciated by those in the art, although one embodiment sequentially utilizes a CRISPR step to knock out B2M, followed by a CRISPR step to knock out CIITA with a final step of a lentivirus to knock in the CD47 functionality, these genes can be manipulated in different orders using different technologies.

As is discussed more fully below, transient expression of reprogramming genes is generally done to generate/induce pluripotent stem cells.

### a. CRISPR Technologies

In one embodiment, the cells are manipulated using clustered regularly interspaced short palindromic repeats)/Cas (“CRISPR”) technologies as is known in the art. CRISPR can be used to generate the starting iPSCs or to generate the HIP cells from the iPSCs. There are a large number of techniques based on CRISPR, see for example Doudna and Charpentier, Science doi: 10.1126/science.1258096, hereby incorporated by reference.

CRISPR techniques and kits are sold commercially.

### b. TALEN Technologies

In some embodiments, the HIP cells of the invention are made using Transcription Activator-Like Effector Nucleases (TALEN) methodologies. TALEN are restriction enzymes combined with a nuclease that can be engineered to bind to and cut practically any desired DNA sequence. TALEN kits are sold commercially.

### c. Zinc Finger Technologies

In one embodiment, the cells are manipulated using Zn finger nuclease technologies. Zn finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms, similar to CRISPR and TALENs.

### d. Viral Based Technologies

There are a wide variety of viral techniques that can be used to generate the HIP cells of the invention (as well as for the original generation of the iPCSs), including, but not limited to, the use of retroviral vectors, lentiviral vectors, adenovirus vectors and Sendai viral vectors. Episomal vectors used in the generation of iPSCs are described below.

### e. Down Regulation of Genes Using Interfering RNA

In other embodiments, genes that encode proteins used in HLA molecules are downregulated by RNAi technologies. RNA interference (RNAi) is a process where RNA molecules inhibit gene expression often by causing specific mRNA molecules to degrade. Two types of RNA molecules—microRNA (miRNA) and small interfering RNA (siRNA)—are central to RNA interference. They bind to the target mRNA molecules and either increase or decrease their activity. RNAi helps cells defend against parasitic nucleic acids such as those from viruses and transposons. RNAi also influences development.

sdRNA molecules are a class of asymmetric siRNAs comprising a guide (antisense) strand of 19-21 bases. They contain a 5′ phosphate, 2′Ome or 2′F modified pyrimidines, and six phosphotioates at the 3′ positions. They also contain a sense strand containing 3′ conjugated sterol moieties, 2 phospotioates at the 3′ position, and 2′Ome modified pyrimidines. Both strands contain 2′ Ome purines with continuous stretches of unmodified purines not exceeding a length of 3. sdRNA is disclosed in U.S. Pat. No. 8,796,443, incorporated herein by reference in its entirety.

For all of these technologies, well known recombinant techniques are used, to generate recombinant nucleic acids as outlined herein. In certain embodiments, the recombinant nucleic acids (either than encode a desired polypeptide, e.g. CD47, or disruption sequences) may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s).

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

### B. Generation of Pluripotent Cells

The invention provides methods of producing non-immunogenic pluripotent cells from pluripotent cells. Thus, the first step is to provide the pluripotent stem cells.

The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al., World J. Stem Cells 7(1):116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogeneous genes. This loss of the episomal vector(s) results in cells that are called “zero footprint” cells. This is desirable as the fewer genetic modifications (particularly in the genome of the host cell), the better. Thus, it is preferred that the resulting hiPSCs have no permanent genetic modifications.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen.

In general, these reprogramming factor genes are provided on episomal vectors such as are known in the art and commercially available. For example, ThermoFisher/Invitrogen sell a sendai virus reprogramming kit for zero footprint generation of hiPSCs, see catalog number A34546. ThermoFisher also sells EBNA-based systems as well, see catalog number A14703.

In addition, there are a number of commercially available hiPSC lines available; see, e.g., the Gibco® Episomal hiPSC line, K18945, which is a zero footprint, viral-integration-free human iPSC cell line (see also Burridge et al, 2011, supra).

In general, as is known in the art, iPSCs are made from non-pluripotent cells such as CD34+ cord blood cells, fibroblasts, etc., by transiently expressing the reprogramming factors as described herein.

For example, successful iPSCs were also generated using only Oct3/4, Sox2 and Klf4, while omitting the C-Myc, although with reduced reprogramming efficiency.

In general, iPSCs are characterized by the expression of certain factors that include KLF4, Nanog, OCT4, SOX2, ESRRB, TBX3, c-Myc and TCL1. New or increased expression of these factors for purposes of the invention may be via induction or modulation of an endogenous locus or from expression from a transgene.

For example, murine iPSCs can be generated using the methods of Diecke et al, Sci Rep. 2015, Jan. 28; 5:8081 (doi:10.1038/srep08081), hereby incorporated by reference in its entirety and specifically for the methods and reagents for the generation of the miPSCs. See also, e.g., Burridge et al., PLoS One, 2011 6(4):18293, hereby incorporated by reference in its entirety and specifically for the methods outlined therein.

In some cases, the pluripotency of the cells is measured or confirmed as outlined herein, for example by assaying for reprogramming factors or by conducting differentiation reactions as outlined herein and in the Examples.

### C. Generation of Hypo-Immunogenic Pluripotent (HIP) Cells

Generating HIP cells from pluripotent cells is done with as few as three genetic changes, resulting in minimal disruption of cellular activity but conferring immunosilencing to the cells.

As discussed herein, one embodiment utilizes a reduction or elimination in the protein activity of MHC I and II (HLA I and II when the cells are human). This can be done by altering genes encoding their component. In one embodiment, the coding region or regulatory sequences of the gene are disrupted using CRISPR. In another embodiment, gene translation is reduced using interfering RNA technologies. The third change is a change in a gene that regulates susceptibility to macrophage phagocytosis, such as CD47, and this is generally a “knock in” of a gene using viral technologies.

In some cases, where CRISPR is being used for the genetic modifications, hiPSC cells that contain a Cas9 construct that enable high efficiency editing of the cell line can be used; see, e.g., the Human Episomal Cas9 iPSC cell line, A33124, from Life Technologies.

1. HLA-I Reduction

The HIP cells of the invention include a reduction in MHC I function (HLA I when the cells are derived from human cells).

As will be appreciated by those in the art, the reduction in function can be accomplished in a number of ways, including removing nucleic acid sequences from a gene, interrupting the sequence with other sequences, or altering the regulatory components of the nucleic acid. For example, all or part of a coding region of the gene of interest can be removed or replaced with “nonsense” sequences, frameshift mutations can be made, all or part of a regulatory sequence such as a promoter can be removed or replaced, translation initiation sequences can be removed or replaced, etc.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A,B,C antibodies that bind to the alpha chain of the human major histocompatibility HLA Class I antigens.

### a. B2M Alteration

In one embodiment, the reduction in HLA-I activity is done by disrupting the expression of the β-2 microglobulin gene in the pluripotent stem cell, the human sequence of which is disclosed herein. This alteration is generally referred to herein as a gene “knock out”, and in the HIP cells of the invention it is done on both alleles in the host cell. Generally the techniques to do both disruptions is the same.

A particularly useful embodiment uses CRISPR technology to disrupt the gene. In some cases, CRISPR technology is used to introduce small deletions/insertions into the coding region of the gene, such that no functional protein is produced, often the result of frameshift mutations that result in the generation of stop codons such that truncated, non-functional proteins are made.

Accordingly, a useful technique is to use CRISPR sequences designed to target the coding sequence of the B2M gene in mouse or the B2M gene in human. After gene editing, the transfected iPSC cultures are dissociated to single cells. Single cells are expanded to full-size colonies and tested for CRISPR edit by screening for presence of aberrant sequence from the CRISPR cleavage site. Clones with deletions in both alleles are picked. Such clones did not express B2M as demonstrated by PCR and did not express HLA-I as demonstrated by FACS analysis (see examples 1 and 6, for example).

Assays to test whether the B2M gene has been inactivated are known and described herein. In one embodiment, the assay is a Western blot of cells lysates probed with antibodies to the B2M protein. In another embodiment, reverse transcriptase polymerase chain reactions (rt-PCR) confirms the presence of the inactivating alteration.

In addition, the cells can be tested to confirm that the HLA I complex is not expressed on the cell surface. This may be assayed by FACS analysis using antibodies to one or more HLA cell surface components as discussed above.

It is noteworthy that others have had poor results when trying to silence the B2M genes at both alleles. See, e.g. Gornalusse et al., Nature Biotech. Doi/10.1038/nbt.3860).

### 2. HLA-II Reduction

In addition to a reduction in HLA I, the HIP cells of the invention also lack MHC II function (HLA II when the cells are derived from human cells).

As will be appreciated by those in the art, the reduction in function can be accomplished in a number of ways, including removing nucleic acid sequences from a gene, adding nucleic acid sequences to a gene, disrupting the reading frame, interrupting the sequence with other sequences, or altering the regulatory components of the nucleic acid. In one embodiment, all or part of a coding region of the gene of interest can be removed or replaced with “nonsense” sequences. In another embodiment, regulatory sequences such as a promoter can be removed or replaced, translation initiation sequences can be removed or replaced, etc.

The successful reduction of the MHC II function (HLA II when the cells are derived from human cells) in the pluripotent cells or their derivatives can be measured using techniques known in the art such as Western blotting using antibodies to the protein, FACS techniques, rt-PCR techniques, etc.

### a. CIITA Alteration

In one embodiment, the reduction in HLA-II activity is done by disrupting the expression of the CIITA gene in the pluripotent stem cell, the human sequence of which is shown herein. This alteration is generally referred to herein as a gene “knock out”, and in the HIP cells of the invention it is done on both alleles in the host cell.

Assays to test whether the CIITA gene has been inactivated are known and described herein. In one embodiment, the assay is a Western blot of cells lysates probed with antibodies to the CIITA protein. In another embodiment, reverse transcriptase polymerase chain reactions (rt-PCR) confirms the presence of the inactivating alteration.

In addition, the cells can be tested to confirm that the HLA II complex is not expressed on the cell surface. Again, this assay is done as is known in the art. Exemplary analyses include Western Blots or FACS analysis using commercial antibodies that bind to human HLA Class II HLA-DR, DP and most DQ antigens as outlined below.

A particularly useful embodiment uses CRISPR technology to disrupt the CIITA gene. CRISPRs were designed to target the coding sequence of the Ciita gene in mouse or the CIITA gene in human, an essential transcription factor for all MHC II molecules. After gene editing, the transfected iPSC cultures were dissociated into single cells. They were expanded to full-size colonies and tested for successful CRISPR editing by screening for the presence of an aberrant sequence from the CRISPR cleavage site. Clones with deletions did not express CIITA as determined by PCR and did not express MHC II/HLA-II as determined by FACS analysis.

### 3. Phagocytosis Reduction

In addition to the reduction of HLA I and II (or MHC I and II), generally using B2M and CIITA knock-outs, the HIP cells of the invention have a reduced susceptibility to macrophage phagocytosis and NK cell killing. The resulting HIP cells “escape” the immune macrophage and innate pathways due to one or more CD47 transgenes.

### a. CD47 Increase

In some embodiments, reduced macrophage phagocytosis and NK cell killing susceptibility results from increased CD47 on the HIP cell surface. This is done in several ways as will be appreciated by those in the art using “knock in” or transgenic technologies. In some cases, increased CD47 expression results from one or more CD47 transgene.

Accordingly, in some embodiments, one or more copies of a CD47 gene is added to the HIP cells under control of an inducible or constitutive promoter, with the latter being preferred. In some embodiments, a lentiviral construct is employed as described herein or known in the art. CD47 genes may integrate into the genome of the host cell under the control of a suitable promoter as is known in the art.

The HIP cell lines were generated from B2M−/− CIITA−/− iPSCs. Cells containing lentivirus vectors expressing CD47 were selected using a Blasticidin marker. The CD47 gene sequence was synthesized and the DNA was cloned into the plasmid Lentivirus pLenti6/V5 with a blasticidin resistance (Thermo Fisher Scientific, Waltham, MA)

In some embodiments, the expression of the CD47 gene can be increased by altering the regulatory sequences of the endogenous CD47 gene, for example, by exchanging the endogenous promoter for a constitutive promoter or for a different inducible promoter. This can generally be done using known techniques such as CRISPR.

Once altered, the presence of sufficient CD47 expression can be assayed using known techniques such as those described in the Examples, such as Western blots, ELISA assays or FACS assays using anti-CD47 antibodies. In general, “sufficiency” in this context means an increase in the expression of CD47 on the HIP cell surface that silences NK cell killing. The natural expression levels on cells is too low to protect them from NK cell lysis once their MHC I is removed.

### 4. Suicide Genes

In some embodiments, the invention provides hypoimmunogenic pluripotent cells that comprise a “suicide gene” or “suicide switch”. These are incorporated to function as a “safety switch” that can cause the death of the hypoimmunogenic pluripotent cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10):1932-1943 (2012), Xu et al., Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In one embodiment, the portion of the Caspase protein is exemplified in SEQ ID NO:6. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 in the instant invention is triggered by the administration of a chemical inducer of dimerization (CID). In some embodiments, the CID is the small molecule drug AP1903. Dimerization causes the rapid induction of apoptosis. (See WO2011146862; Stasi et al, N. Engl. J Med 365; 18 (2011); Tey et al., Biol. Blood Marrow Transplant. 13:913-924 (2007), each of which are incorporated by reference herein in their entirety.)

### 5. CD16, CD32, or CD64 Expression

The cells of the invention have a reduced susceptibility to ADCC and CDC resulting from increased CD16, CD32, or CD64 expression. The resulting cells sequester antibodies due to the increased expression. In one embodiment, the cells comprise one or more CD16, CD32, or CD64 transgenes.

### a. CD16, CD32, or CD64 Increase

In some embodiments, reduced ADCC or CDC susceptibility results from increased CD16, CD32, or CD64 on the cell surface. This is done in several ways as will be appreciated by those in the art using “knock in” or transgenic technologies. In some cases, increased CD16, CD32, or CD64 expression results from one or more transgenes.

Accordingly, in some embodiments, one or more copies of a CD16, CD32, or CD64 gene is added to the cells under control of an inducible or constitutive promoter, with the latter being preferred. In some embodiments, a lentiviral construct is employed as described herein or known in the art. The genes may integrate into the genome of the host cell under the control of a suitable promoter as is known in the art.

Cells containing lentivirus vectors expressing CD16, CD32, or CD64 are selected using a Blasticidin marker. The gene sequence is synthesized and the DNA may be cloned, for instance, into the plasmid Lentivirus pLenti6/V5 with a blasticidin resistance (Thermo Fisher Scientific, Waltham, MA)

In some embodiments, the expression of the gene can be increased by altering the regulatory sequences of the endogenous CD16, CD32, or CD64 gene, for example, by exchanging the endogenous promoter for a constitutive promoter or for a different inducible promoter. This can generally be done using known techniques such as CRISPR.

Once altered, the presence of sufficient expression can be assayed using known techniques such as those described in the Examples, such as Western blots, ELISA assays or FACS assays using anti-CD16, CD32, or CD64 antibodies. In general, “sufficiency” in this context means an increase in expression the cell surface that sequesters antibodies and inhibis ADCC or CDC.

### 6. Assays for HIP Phenotypes and Retention of Pluripotency

Once the HIP cells have been generated, they may be assayed for their hypo-immunogenicity and/or retention of pluripotency as is generally described herein and in the examples.

For example, hypo-immunogenicity are assayed using a number of techniques One exemplary technique includes transplantation into allogeneic hosts and monitoring for HIP cell growth (e.g. teratomas) that escape the host immune system. HIP derivatives are transduced to express luciferase and can then followed using bioluminescence imaging. Similarly, the T cell and/or B cell response of the host animal to the HIP cells are tested to confirm that the HIP cells do not cause an immune reaction in the host animal. T cell function is assessed by Elispot, Elisa, FACS, PCR, or mass cytometry (CYTOF). B cell response or antibody response is assessed using FACS or luminex. Additionally, or alternatively, the cells may be assayed for their ability to avoid innate immune responses, e.g. NK cell killing. NK cell lytolytic activity is assessed in vitro or in vivo using techniques known in the art.

Similarly, the retention of pluripotency is tested in a number of ways. In one embodiment, pluripotency is assayed by the expression of certain pluripotency-specific factors as generally described herein. Additionally or alternatively, the HIP cells are differentiated into one or more cell types as an indication of pluripotency.

### D. Generation of HIPO− CD16, CD32, or CD64-Overexpressing Cells

In some aspects of the invention, the HIP cells generated as above will already be HIPO− cells because the process will have started with pluripotent cells having an O-blood type.

Other aspects of the invention involve the enzymatic conversion of A and B antigens. In preferred aspects, the B antigen is converted to O using an enzyme. In more preferred aspects, the enzyme is an α-galactosidase. This enzyme eliminates the terminal galactose residue of the B antigen. Other aspects of the invention involve the enzymatic conversion of A antigen to O. In preferred aspects, the A antigen is converted to O using an α-N-acetylgalactosaminidase. Enzymatic conversion is discussed, e.g., in Olsson et al., Transfusion Clinique et Biologique 11:33-39 (2004); U.S. Pat. Nos. 4,427,777, 5,606,042, 5,633,130, 5,731,426, 6,184,017, 4,609,627, and 5,606,042; and Int′l Pub. No. WO9923210, each of which are incorporated by reference herein in their entirety.

Other embodiments of the invention involve genetically engineering the cells by knocking out the ABO gene Exon 7 or silencing the SLC14A1 (JK) gene. Other embodiments of the invention involve knocking out the C and E antigens of the Rh blood group system (RH), K in the Kell system (KEL), Fya and Fy3 in the Duffy system (FY), Jkb in the Kidd system (JK), or U and S in the MNS blood group system. Any knockout methodology known in the art or described herein, such as CRISPR, talens, or homologous recombination, may be employed.

### E. Preferred Embodiments of the Invention

The CD16, CD32, or CD64 overexpressing HIP, HIPO−, iPSC, iPSCO−, ESC, or ESCO− cells, or derivatives thereof, of the invention may be used to treat, for example, Type 1 diabetes, cardiac diseases, neurological diseases, cancer, blindness, vascular diseases, and others that respond to regenerative medicine therapies. In particular, the invention contemplates using the cells for differentiation into any cell type. Thus, provided herein are cells that have enhanced CD16, CD32, or CD64 expression and exhibit pluripotency but do not result in a host ADCC or CDC response when transplanted into an allogeneic host such as a human patient, either as pluripotent cells or as the differentiated products of them.

In one aspect, the cells of the present invention comprise a nucleic acid encoding a chimeric antigen receptor (CAR), wherein CD16, CD32, or CD64 expression has been increased. The CAR can comprise an extracellular domain, a transmembrane domain, and an intracellular signaling domain.

In some embodiments, the extracellular domain binds to an antigen selected from the group consisting of CD19, CD20, CD22, CD38, CD123, CS1, CD171, BCMA, MUC16, ROR1, and WTT. In certain embodiments, the extracellular domain comprises a single chain variable fragment (scFv). In some embodiments, the transmembrane domain comprises CD3ζ, CD4, CD8a, CD28, 4-1BB, OX40, ICOS, CTLA-4, PD-1, LAG-3, and BTLA. In certain embodiments, the intracellular signaling domain comprises CD3ζ, CD28, 4-1BB, OX40, ICOS, CTLA-4, PD-1, LAG-3, and BTLA.

In certain embodiments, the CAR comprises an anti-CD19 scFv domain, a CD28 transmembrane domain, and a CD3 zeta signaling intracellular domain. In some embodiments, the CAR comprises anti-CD19 scFv domain, a CD28 transmembrane domain, a 4-1BB signaling intracellular domain, and a CD3 zeta signaling intracellular domain.

In another aspect of the invention, provided is an isolated CAR-T overexpressing cell CD16, CD32, or CD64 produced by in vitro differentiation of any one of the cells described herein. In some embodiments, the cell is a cytotoxic hypoimmune CAR-T cell.

In various embodiments, the in vitro differentiation comprises culturing the cell carrying a CAR construct in a culture media comprising one or more growth factors or cytokines selected from the group consisting of bFGF, EPO, Flt3L, IGF, IL-3, IL-6, IL-15, GM-CSF, SCF, and VEGF. In some embodiments, the culture media further comprises one or more selected from the group consisting of a BMP activator, a GSK3 inhibitor, a ROCK inhibitor, a TGFβ receptor/ALK inhibitor, and a NOTCH activator.

In particular embodiments, the isolated CAR-T cell of the invention produced by in vitro differentiation is used as a treatment of cancer.

In another aspect of the invention, provided is a method of treating a patient with cancer by administering a composition comprising a therapeutically effective amount of any of the isolated CAR-T cells described herein. In some embodiments, the composition further comprises a therapeutically effective carrier.

In some embodiments, the administration step comprises intravenous administration, subcutaneous administration, intranodal administration, intratumoral administration, intrathecal administration, intrapleural administration, and intraperitoneal administration. In certain instances, the administration further comprises a bolus or by continuous perfusion.

In some embodiments, the cancer is a blood cancer selected from the group consisting of leukemia, lymphoma, and myeloma. In various embodiments, the cancer is a solid tumor cancer or a liquid tumor cancer.

In another aspect, the present invention provides a method of making any one of the isolated CAR-T/CD16, CD32, or CD64 cells described herein. The method includes in vitro differentiating of any one of the cells of the invention wherein in vitro differentiating comprises culturing them in a culture media comprising one or more growth factors or cytokines selected from the group consisting of bFGF, EPO, Flt3L, IGF, IL-2, IL-3, IL-6, IL-7, IL-15, GM-CSF, SCF, and VEGF. In some embodiments, the culture media further comprises one or more selected from the group consisting of a BMP activator, a GSK3 inhibitor, a ROCK inhibitor, a TGFβ receptor/ALK inhibitor, and a NOTCH activator.

In some embodiments, the in vitro differentiating comprises culturing the HIPO-cells on feeder cells. In various embodiments, the in vitro differentiating comprises culturing in simulated microgravity. In certain instances, the culturing in simulated microgravity is for at least 72 hours.

In some aspects, provided herein is an isolated, engineered hypoimmune cardiac cell (hypoimmunogenic cardiac cell) differentiated from a cell with enhanced CD16, CD32, or CD64 expression.

In some aspects, provided herein is a method of treating a patient suffering from a heart condition or disease. The method comprises administering a composition comprising a therapeutically effective amount of a population of any one of the isolated, engineered hypoimmune cardiac cells derived from cells of the invention as described herein. In some embodiments, the composition further comprises a therapeutically effective carrier.

In some embodiments, the administration comprises implantation into the patient's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the heart condition or disease is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.

In some aspects, provided herein is a method of producing a population of hypoimmune cardiac cells from a population of HIPO−/CD16, CD32, or CD64 cells by in vitro differentiation, wherein endogenous 3-2 microglobulin (B2M) gene activity and endogenous class II transactivator (CIITA) gene activity have been eliminated and CD47 expression has been increased in the HIPO-cells. The method comprises: (a) culturing a population of HIPO-cells in a culture medium comprising a GSK inhibitor; (b) culturing the population of HIPO-cells in a culture medium comprising a WNT antagonist to produce a population of pre-cardiac cells; and (c) culturing the population of pre-cardiac cells in a culture medium comprising insulin to produce a population of hypoimmune cardiac cells.

In some embodiments, the GSK inhibitor is CHIR-99021, a derivative thereof, or a variant thereof. In some instances, the GSK inhibitor is at a concentration ranging from about 2 μM to about 10 μM. In some embodiments, the WNT antagonist is IWR1, a derivative thereof, or a variant thereof. In some instances, the WNT antagonist is at a concentration ranging from about 2 μM to about 10 μM.

In some aspects, provided herein is an isolated, engineered CD16, CD32, or CD64-overexpressing endothelial cell differentiated from HIPO− cells. In other aspects, the isolated, engineered endothelial cell of the invention is selected from the group consisting of a capillary endothelial cell, vascular endothelial cell, aortic endothelial cell, brain endothelial cell, and renal endothelial cell.

In some aspects, provided herein is a method of treating a patient suffering from a vascular condition or disease. In some embodiments, the method comprises administering a composition comprising a therapeutically effective amount of a population of isolated, engineered endothelial cells of the invention.

The method comprises administering a composition comprising a therapeutically effective amount of a population of any one of the isolated, engineered CD16, CD32, or CD64-overexpressing endothelial cells described herein. In some embodiments, the composition further comprises a therapeutically effective carrier or excipient. In some embodiments, the administration comprises implantation into the patient's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the vascular condition or disease is selected from the group consisting of vascular injury, cardiovascular disease, vascular disease, ischemic disease, myocardial infarction, congestive heart failure, hypertension, ischemic tissue injury, limb ischemia, stroke, neuropathy, and cerebrovascular disease.

In some aspects, provided herein is a method of producing a population of hypoimmune endothelial cells from a population of CD16, CD32, or CD64-overexpressing cells by in vitro differentiation, wherein endogenous J-2 microglobulin (B2M) gene activity and endogenous class II transactivator (CIITA) gene activity have been eliminated and CD47 expression has been increased in the HIPO-cells. The method comprises: (a) culturing a population of HIPO-cells in a first culture medium comprising a GSK inhibitor; (b) culturing the population of HIPO-cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of hypoimmune endothelial cells.

In some embodiments, the GSK inhibitor is CHIR-99021, a derivative thereof, or a variant thereof. In some instances, the GSK inhibitor is at a concentration ranging from about 1 μM to about 10 μM. In some embodiments, the ROCK inhibitor is Y-27632, a derivative thereof, or a variant thereof. In some instances, the ROCK inhibitor is at a concentration ranging from about 1 μM to about 20 μM. In some embodiments, the ALK inhibitor is SB-431542, a derivative thereof, or a variant thereof. In some instances, the ALK inhibitor is at a concentration ranging from about 0.5 μM to about 10 μM.

In some embodiments, the first culture medium comprises from 2 μM to about 10 μM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 μM Y-27632 and 1 μM SB-431542. In certain embodiments, the third culture medium further comprises VEGF and bFGF. In particular instances, the first culture medium and/or the second medium is absent of insulin.

In some aspects, provided herein is an isolated, engineered hypoimmune dopaminergic neuron (DN) differentiated from a CD16, CD32, or CD64-overexpressing cell. In some embodiments, the endogenous 3-2 microglobulin (B2M) gene activity and endogenous class II transactivator (CIITA) gene activity have been eliminated, CD47 expression has been increased, the neuron is blood type O and Rh—.

In some embodiments, the isolated dopaminergic neuron is selected from the group consisting of a neuronal stem cell, neuronal progenitor cell, immature dopaminergic neuron, and mature dopaminergic neuron.

In some aspects, provided herein is a method of treating a patient suffering from a neurodegenerative disease or condition. In some embodiments, the method comprises administering a composition comprising a therapeutically effective amount of a population of any one of the isolated hypoimmune dopaminergic neurons. In some embodiments, the composition further comprises a therapeutically effective carrier. In some embodiments, the population of the isolated hypoimmune dopaminergic neurons is on a biodegradable scaffold. The administration may comprise transplantation or injection. In some embodiments, the neurodegenerative disease or condition is selected from the group consisting of Parkinson's disease, Huntington disease, and multiple sclerosis.

In some aspects, provided herein is a method of producing a population of CD16, CD32, or CD64-overexpressing dopaminergic neurons by in vitro differentiation. In some embodiments, the endogenous β-2 microglobulin (B2M) gene activity and endogenous class II transactivator (CIITA) gene activity have been eliminated, CD47 expression has been increased, the blood group is O and Rh—. In some embodiments, the method comprises (a) culturing the population of cells in a first culture medium comprising one or more factors selected from the group consisting of sonic hedgehog (SHH), BDNF, EGF, bFGF, FGF8, WNT1, retinoic acid, a GSK3β inhibitor, an ALK inhibitor, and a ROCK inhibitor to produce a population of immature dopaminergic neurons; and (b) culturing the population of immature dopaminergic neurons in a second culture medium that is different than the first culture medium to produce a population of dopaminergic neurons.

In some embodiments, the GSK3 inhibitor is CHIR-99021, a derivative thereof, or a variant thereof. In some instances, the GSK3 inhibitor is at a concentration ranging from about 2 μM to about 10 μM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

In some embodiments, the method also comprises isolating the population of hypoimmune dopaminergic neurons from non-dopaminergic neurons. In some embodiments, the method further comprises cryopreserving the isolated population of hypoimmune dopaminergic neurons.

In some aspects, provided herein is an isolated engineered hypoimmune pancreatic islet cell differentiated from a CD16, CD32, or CD64-overexpressing cell. In some embodiments, the endogenous J-2 microglobulin (B2M) gene activity and endogenous class II transactivator (CIITA) gene activity have been eliminated, CD47 expression has been increased, the blood type is O and Rh—.

In some embodiments, the isolated hypoimmune pancreatic islet cell is selected from the group consisting of a pancreatic islet progenitor cell, immature pancreatic islet cell, and mature pancreatic islet cell.

In some aspects, provided herein is a method of treating a patient suffering from diabetes. The method comprises administering a composition comprising a therapeutically effective amount of a population of any one of the isolated CD16, CD32, or CD64-overexpressing pancreatic islet cells described herein. In some embodiments, the composition further comprises a therapeutically effective carrier. In some embodiments, the population of the isolated hypoimmune pancreatic islet cells is on a biodegradable scaffold. In some instances, the administration comprises transplantation or injection.

In some aspects, provided herein is a method of producing a population of hypoimmune pancreatic islet cells from a population of CD16, CD32, or CD64-overexpressing cells by in vitro differentiation. In some embodiments, the endogenous 3-2 microglobulin (B2M) gene activity and endogenous class II transactivator (CIITA) gene activity have been eliminated, CD47 expression has been increased, the blood type is O and Rh— in the HIPO− cells. The method comprises: (a) culturing the population of CD16, CD32, or CD64-overexpressing cells in a first culture medium comprising one or more factors selected from the group consisting insulin-like growth factor (IGF), transforming growth factor (TGF), fibroblast growth factor (EGF), epidermal growth factor (EGF), hepatocyte growth factor (HGF), sonic hedgehog (SHH), and vascular endothelial growth factor (VEGF), transforming growth factor-β (TGFβ) superfamily, bone morphogenic protein-2 (BMP2), bone morphogenic protein-7 (BMP7), a GSK3β inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of hypoimmune pancreatic islet cells.

In some embodiments, the GSK inhibitor is CHIR-99021, a derivative thereof, or a variant thereof. In some instances, the GSK inhibitor is at a concentration ranging from about 2 μM to about 10 μM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

In some embodiments, the method also comprises isolating the population of CD16, CD32, or CD64-overexpressing pancreatic islet cells from non-pancreatic islet cells. In some embodiments, the method further comprises cryopreserving the isolated population of hypoimmune pancreatic islet cells.

In some aspects, provided herein is an isolated, engineered hypoimmune retinal pigmented epithelium (RPE) cell differentiated from a CD16, CD32, or CD64-overexpressing cell, wherein endogenous 3-2 microglobulin (B2M) gene activity and endogenous class II transactivator (CIITA) gene activity have been eliminated, CD47 expression has been increased, the blood type is O and Rh—.

In some embodiments, the isolated hypoimmune RPE cell is selected from the group consisting of a RPE progenitor cell, immature RPE cell, mature RPE cell, and functional RPE cell.

In some aspects, provided herein is a method of treating a patient suffering from an ocular condition. The method comprises administering a composition comprising a therapeutically effective amount of a population of any one of a population of the isolated CD16, CD32, or CD64-overexpressing RPE cells described herein. In some embodiments, the composition further comprises a therapeutically effective carrier. In some embodiments, the population of the isolated hypoimmune RPE cells is on a biodegradable scaffold. In some embodiments, the administration comprises transplantation or injection to the patient's retina. In some embodiments, the ocular condition is selected from the group consisting of wet macular degeneration, dry macular degeneration, juvenile macular degeneration, Leber's Congenital Ameurosis, retinitis pigmentosa, and retinal detachment.

In some aspects, provided herein is a method of producing a population of CD16, CD32, or CD64-overexpressing retinal pigmented epithelium (RPE) cells from a population of cells by in vitro differentiation. In some embodiments, the endogenous 3-2 microglobulin (B2M) gene activity and endogenous class II transactivator (CIITA) gene activity have been eliminated and CD47 expression has been increased in the HIPO-cells. The method comprises: (a) culturing the population of HIPO-cells in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of hypoimmune RPE cells.

In some embodiments, the ALK inhibitor is SB-431542, a derivative thereof, or a variant thereof. In some instances, the ALK inhibitor is at a concentration ranging from about 2 μM to about 10 μM. In some embodiments, the ROCK inhibitor is Y-27632, a derivative thereof, or a variant thereof. In some instances, the ROCK inhibitor is at a concentration ranging from about 1 μM to about 10 μM.

In some embodiments, the first culture medium and/or second culture medium are absent of animal serum.

In some embodiments, the method further comprises isolating the population of hypoimmune RPE cells from non-RPE cells. In some embodiments, the method further comprises cryopreserving the isolated population of hypoimmune RPE cells.

In one aspect, human pluripotent stem cells (PSCs) resist ADCC or CDC by CD16, CD32, or CD64-overexpression. In some embodiments, they are hypoimmune pluripotent stem cells (hiPSC). They are rendered hypo-immunogenic by a) the disruption of the B2M gene at each allele (e.g. B2M −/−), b) the disruption of the CIITA gene at each allele (e.g. CIITA −/−), and c) by the overexpression of the CD47 gene (CD47+, e.g. through introducing one or more additional copies of the CD47 gene or activating the genomic gene). This renders the hiPSC population B2M−/− CIITA −/− CD47tg. In a preferred aspect, the cells are non-immunogenic. In another embodiment, the HIP cells are rendered non-immunogenic B2M−/− CIITA −/− CD47tg as described above but are further modified by including an inducible suicide gene that is induced to kill the cells in vivo when required. In other aspects, CD16, CD32, or CD64-overexpressing HIPO cells are created when HIP cells are rendered blood type O by knocking out the ABO gene Exon 7 or silencing the SLC14A1 (JK) gene and the cells are rendered Rh— by knocking out the C and E antigens of the Rh blood group system (RH), K in the Kell system (KEL), Fya and Fy3 in the Duffy system (FY), Jkb in the Kidd system (JK), or U and S in the MNS blood group system.

### F. Maintenance of HIPO−/CD16, CD32, or CD64 Cells

Once generated, the HIPO-CD16, CD32, or CD64 cells can be maintained in an undifferentiated state as is known for maintaining iPSCs. For example, HIP cells are cultured on Matrigel using culture media that prevents differentiation and maintains pluripotency.

### G. Differentiation of HIPO−/CD16, CD32, or CD64 Cells

The invention provides HIPO−/CD16, CD32, or CD64 cells that are differentiated into different cell types for subsequent transplantation into subjects. As will be appreciated by those in the art, the methods for differentiation depend on the desired cell type using known techniques. The cells are differentiated in suspension and then put into a gel matrix form, such as matrigel, gelatin, or fibrin/thrombin forms to facilitate cell survival. Differentiation is assayed as is known in the art, generally by evaluating the presence of cell-specific markers.

In some embodiments, the HIPO−/CD16, CD32, or CD64 cells are differentiated into hepatocytes to address loss of the hepatocyte functioning or cirrhosis of the liver. There are a number of techniques that can be used to differentiate HIPO− cells into hepatocytes; see for example Pettinato et al., doi:10.1038/spre32888, Snykers et al., Methods Mol Biol 698:305-314 (2011), Si-Tayeb et al, Hepatology 51:297-305 (2010) and Asgari et al., Stem Cell Rev (:493-504 (2013), all of which are hereby expressly incorporated by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

In some embodiments, the HIPO−/CD16, CD32, or CD64 cells are differentiated into beta-like cells or islet organoids for transplantation to address type I diabetes mellitus (T1DM). Cell systems are a promising way to address T1DM, see, e.g., Ellis et al., doi/10.1038/nrgastro.2017.93, incorporated herein by reference. Additionally, Pagliuca et al. reports on the successful differentiation of R-cells from hiPSCs (see doi/10.106/j.cell.2014.09.040, hereby incorporated by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; (doi:10.1038/nm.4030, hereby incorporated by reference in its entirety and in particular for the methods and reagents outlined there for the large-scale production of functional human β cells from human pluripotent stem cells).

Differentiation is assayed as is known in the art, generally by evaluating the presence of β cell associated or specific markers, including but not limited to, insulin. Differentiation can also be measured functionally, such as measuring glucose metabolism, see generally Muraro et al, doi:10.1016/j.cels.2016.09.002, hereby incorporated by reference in its entirety, and specifically for the biomarkers outlined there.

Once the dHIPO−/CD16, CD32, or CD64 beta cells are generated, they can be transplanted (either as a cell suspension or within a gel matrix as discussed herein) into the portal vein/liver, the omentum, the gastrointestinal mucosa, the bone marrow, a muscle, or subcutaneous pouches.

In some embodiments, the HIPO−/CD16, CD32, or CD64 cells are differentiated into retinal pigment epithelium (RPE) to address sight-threatening diseases of the eye. Human pluripotent stem cells have been differentiated into RPE cells using the techniques outlined in Kamao et al., Stem Cell Reports 2014:2:205-18, hereby incorporated by reference in its entirety and in particular for the methods and reagents outlined there for the differentiation techniques and reagents; see also Mandai et al., doi:10.1056/NEJMoa1608368, also incorporated in its entirety for techniques for generating sheets of RPE cells and transplantation into patients.

Differentiation can be assayed as is known in the art, generally by evaluating the presence of RPE associated and/or specific markers or by measuring functionally. See for example Kamao et al., doi:10.1016/j.stemcr.2013.12.007, hereby incorporated by reference in its entirety and specifically for the markers outlined in the first paragraph of the results section.

In some embodiments, the HIPO−/CD16, CD32, or CD64 cells are differentiated into cardiomyocytes to address cardiovascular diseases. Techniques are known in the art for the differentiation of hiPSCs to cardiomyoctes and discussed in the Examples. Differentiation can be assayed as is known in the art, generally by evaluating the presence of cardiomyocyte associated or specific markers or by measuring functionally; see for example Loh et al., doi:10.1016/j.cell.2016.06.001, hereby incorporated by reference in its entirety and specifically for the methods of differentiating stem cells including cardiomyocytes.

In some embodiments, the HIPO−/CD16, CD32, or CD64 cells are differentiated into endothelial colony forming cells (ECFCs) to form new blood vessels to address peripheral arterial disease. Techniques to differentiate endothelial cells are known. See, e.g., Prasain et al., doi:10.1038/nbt.3048, incorporated by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the HIPO−/CD16, CD32, or CD64 cells are differentiated into thyroid progenitor cells and thyroid follicular organoids that can secrete thyroid hormones to address autoimmune thyroiditis. Techniques to differentiate thyroid cells are known the art. See, e.g. Kurmann et al., doi:10.106/j.stem.2015.09.004, hereby expressly incorporated by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

### H. Transplantation of Differentiated HIPO−/CD16, CD32, or CD64 Cells

As will be appreciated by those in the art, the differentiated HIPO−/CD16, CD32, or CD64 derivatives are transplated using techniques known in the art that depends on both the cell type and the ultimate use of these cells. In general, the cells of the invention are transplanted either intravenously or by injection at particular locations in the patient. When transplanted at particular locations, the cells may be suspended in a gel matrix to prevent dispersion while they take hold.

In order that the invention described herein may be more fully understood, the following examples are set forth. It should be understood that these examples are for illustrative purposes only and are not to be construed as limiting this invention in any manner.

### VIII. EXAMPLES

HIP and HIPO− cells are generated as disclosed in WO2018/132783, PCT/US19/42123, PCT/US19/42117, and Prov. Appl. Nos. 62/698,973, 62/698,978, 62/698,981, 62/698,984, 62/846,399, 62/855,499, each of which are incorporated by reference herein in their entirety.

### A. Example 1: CD64 Protected Macrophages from NK Cell ADCC Killing

Constitutive CD64 expression was shown to protect macrophages, which naturally express CD52, from NK cell ADCC when challenged with an anti-CD52 antibody. Hypoimmune macrophages (derived from B2M^{-/-} CIITA^{-/-} CD47 tg HIP iPSCs) that consitutively express CD52 were plated on the XCelligence platform for in vitro impedance assays (ACEA BioSciences, San Diego, CA). They attached to plastic dishes and formed confluent layers. The FDA-approved humanized IgG antibody alemtuzumab (Bio-Rad, Hercules, CA, Catalog No. MCA6101) was added at a concentration of 0.001, 0.01, 0.1, or 1.0 μg/ml. Alemtuzumab is an anti-CD52 antibody capable of mediating both ADCC and CDC. Then, NK cells were added to the assay. Macrophage killing was assessed by measuring the impedance between electrodes on the special 96-well E-plates (ACEA BioSciences).

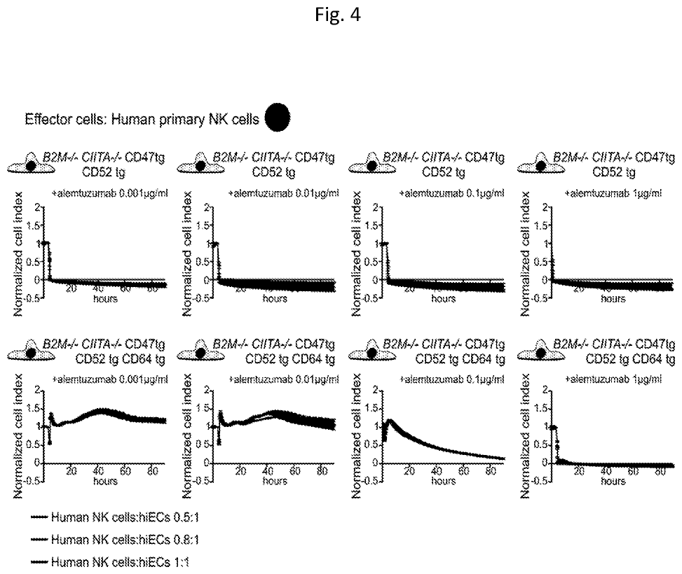

FIG. 3, upper row, shows that high concentrations of alemtuzumab were necessary for NK cell-mediated ADCC and macrophages were killed only at 1.0 μg/ml. Without wishing to be bound by theory, at the highest alemtuzumab concentration, it is postulated that all the CD64 receptors were occupied and not available to sequester enough alentuzumab Fe domains to alleviate ADCC. When a blocking antibody against CD64 (Thermo Fisher Scientific, Carlsbad, CA, Clone 10.1, Catalog No. MA1-10270) was added to the assay at a concentration of 5 μg/ml (lower row), the ADCC killing was enhanced. Target cell killing started already at an alemtuzumab concentration of 0.001 μg/ml. With increasing alemtuzumab concentrations, macrophage killing in the presence of CD64 blocking antibodies became more efficient.

### B. Example 2: CD64 Protected Macrophages from CDC Killing

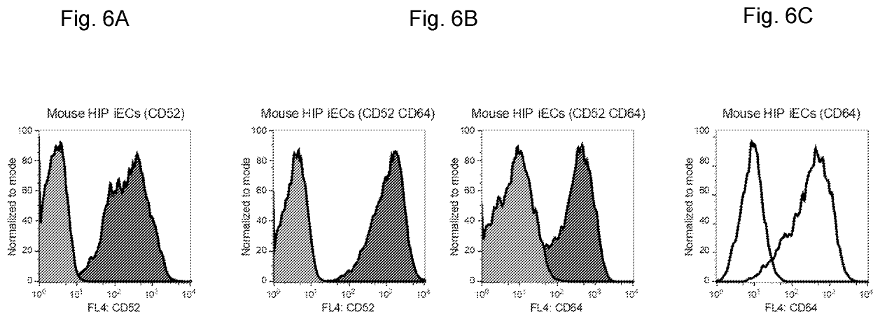

CD64 was shown to protect CD52+ macrophages from CDC when challenged with an anti-CD52 antibody. Macrophages were plated on Xcelligence plates and challenged with alemtuzumab as in Example 1. Serum from a blood type A donor (compatible to the blood type of the target cells) was added to avoid additional blood-type antibodies in the assay. All the complement components, however, were present. The first row of FIG. 5 shows that macrophages have some constitutive protection against CDC because the lower concentrations of alemtuzumab did not lead to profound killing. CD64 protection was clearly shown at 0.001 and 0.01 μg/ml of alemtuzumab. This is because the anti-CD64 antibody caused significant CDC death. (FIG. 5, lower row). This shows that macrophages have a constitutive protection against CDC through their CD64 expression that can be overwhelmed at high antibody concentrations.

### C. Example 3: CD64 Protected Engineered Endothelial Cells from ADCC Killing by NK Cells

CD64 was shown to protect CD52+ endothelial cells from ADCC when challenged with anti-CD52 antibodies. Endothelial cells were differentiated from HIP Cells as follows. The differentiation protocol was initiated at 60% HIP confluency, then the medium was changed to RPMI-1640 containing 2% B-27 minus insulin (both Gibco, a Thermo Fisher Scientific Brand) and 5 μM CHIR-99021 (Selleckchem, Munich, Germany). On day 2, the medium was changed to reduced medium: RPMI-1640 containing 2% B-27 minus insulin (Gibco) and 2 μM CHIR-99021 (Selleckchem). From culture days 4 to 7, cells were exposed to RPMI-1640 EC medium, RPMI-1640 containing 2% B-27 minus insulin plus 50 ng/ml human vascular endothelial growth factor (VEGF; R&D Systems, Minneapolis, MN), 10 ng/ml human fibroblast growth factor basic (FGFb; R&D Systems), 10 μM Y-27632 (Sigma-Aldrich, St. Louis, MO), and 1 μM SB 431542 (Sigma-Aldrich). Endothelial cell clusters were visible from day 7 and cells were maintained in Endothelial Cell Basal Medium 2 (PromoCell, Heidelberg, Germany) plus supplements, 10% FCS hi (Gibco), 1% pen/strep, 25 ng/ml VEGF, 2 ng/ml FGFb, 10 μM Y-27632 (Sigma-Aldrich), and 1 μM SB 431542 (Sigma-Aldrich). The differentiation protocol was completed after 14 days; undifferentiated cells detached during the differentiation process. TrypLE Express (Gibco) was used for passaging the cells 1:3 every 3 to 4 days. Then the HIP-derived epithelial cells were transformed with a lentiviral vector that expresses CD52: For transfection experiments, 1.5×10⁵ human B2M^{-/-}CIITA^{-/-} CD47 tg ECs were plated per well of a 6-well plate. The cells were incubated overnight at 37° C. in a cell incubator. The next day, transfection was performed using Fugene (Promaga, Fitchburg, WI) and 5 μg of CD52 expression particles (Origene, Rockville, MD, CAT #: RC200493L2V) at a ratio of 3:2. The transfection reagent solution was pipetted to OptiMEM (Gibco), mixed, and incubated for 10 minutes at room temperature. The DNA transfection complex was in to 2 ml cell medium. After 24 h, the transfection was stopped and cells were grown for a further 48 h in endothelial cell medium (Gibco). Successful transfection was confirmed by flow cytometry analysis and CD52 positive cells were enriched via FACS sorting on FACS Aria (CD52 APC: clone HI186, Biolegend).

A subset of the CD52+B2M^{-/-}CIITA^{-/-} CD47 tg ECs were further transformed with a lentivirus to express CD64 using the same protocol. The transfection was performed using Fugene (Promaga) and 5 μg of CD64 expression plasmids (Origene, Rockville, MD, CAT #: RC207487L2V) in a ratio of 3:2. The transfection was stopped as outlined above and cells were expanded for another 48 h. Successful transfection was confirmed by flow cytometry analysis and CD64 positive cells were enriched via FACS sorting on FACS Aria (CD64 PE: clone 10.1, BD). This CD52/CD64 double positive population was then culture expanded.

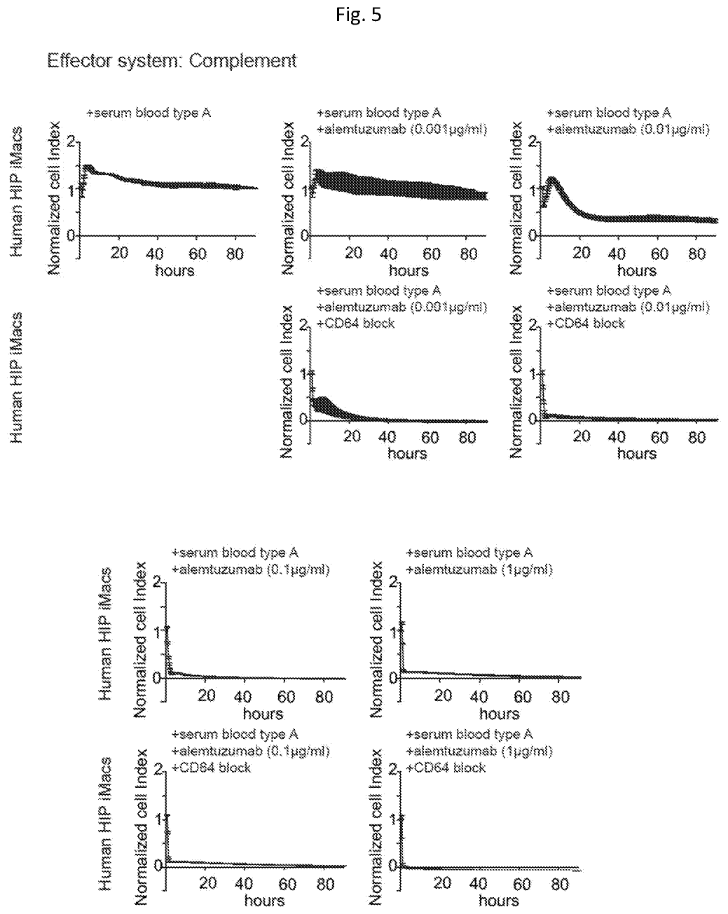

Both EC lines were assessed in XCelligence assays. FIG. 4 on the top row shows that the CD52 confered significant ADCC susceptibility. The CD64-expressing CD52 tg ECs in the lower row were significantly better protected against ADCC, especially at lower alemtuzumab concentrations.

Using a different assay, CD64 was again shown to protect CD52+ mouse endothelial cells from ADCC when challenged with anti-CD52 antibodies. Endothelial cells were differentiated from mouse HIP iPSCs as follows. Mouse HIP iPSC were plated on gelatin in 6-well plates and maintained in mouse iPSC media. After the cells reached 60% confluency, the differentiation was started and media was changed to RPMI-1640 containing 2% B-27 minus Insulin (both Gibco) and 5 μM CHIR-99021 (Selleckchem, Munich, Germany). On day 2, the media was changed to reduced media: RPMI-1640 containing 2% B-27 minus Insulin (both Gibco) and 2 μM CHIR-99021 (Selleckchem). From day 4 to day 7, cells were exposed to RPMI-1640 EC media, RPMI-1640 containing 2% B-27 minus Insulin plus 50 ng/mL mouse vascular endothelial growth factor (mVEGF; R&D Systems, Minneapolis, MN), 10 ng/mL mouse fibroblast growth factor basic (mFGFb; R&D Systems), 10 μM Y-27632 (Sigma-Aldrich, Saint Louis, MO), and 1 μM SB 431542 (Sigma-Aldrich). Endothelial cell clusters were visible from day 7 and cells were maintained in EGM-2 SingleQuots media (Lonza) plus 10% FCS hi (Gibco), 25 ng/mL mVEGF, 2 ng/mL mFGFb, 10 μM Y-27632 (Sigma-Aldrich), and 1 μM SB 431542. The differentiation process was completed after 21 days and undifferentiated cells detached during the differentiation process. For purification, cells went through MACS purification according the manufactures' protocol using anti-CD15 mAb-coated magnetic microbeads (Miltenyi, Auburn, CA) for negative selection. The highly purified miECs in the flow-through were cultured in EGM-2 SingleQuots media plus supplements and 10% FCS hi. TrypLE was used for passaging the cells 1:3 every 3 to 4 days. Their phenotype was confirmed by immunofluorescence (IF) for CD31 (ab28364, Abcam), and VE-Cadherin (sc-6458, Santa Cruz Biotechnology, Santa Cruz, CA).

Some HIP-derived mouse iECs were transduced with a lentiviral vector that expressed CD52: For transfection experiments, 1.5×10⁵ mouse HIP iECs were plated per well in a 6-well plate. The cells were incubated overnight at 37° C. in a cell incubator. On the next day, a transfection was performed using Fugene (Promaga, Fitchburg, WI) and 5 μg of CD52 expression particles (Origene, Rockville, MD, CAT #: RC200493L2V) in a ratio of 3:2. The transfection reagent solution was pipetted to OptiMEM (Gibco), mixed, and incubated for 10 minutes at room temperature. The DNA transfection complex was added to 2 ml of cell medium. After 24 h, the transfection was stopped, and cells were grown for a further 48 h in endothelial cell medium (Gibco).

A subset of the mouse HIP iECs (CD52) were further transformed with a lentivirus to express CD64 using the same protocol. The transfection was performed using Fugene (Promaga) and 5 μg of CD64 expression plasmids (Origene, Rockville, MD, CAT #: RC207487L2V) in a ratio of 3:2. The transfection was stopped as outlined above and cells were expanded for another 48 h.

Some HIP-derived mouse iECs were transduced to only express CD64 according to the protocol above.

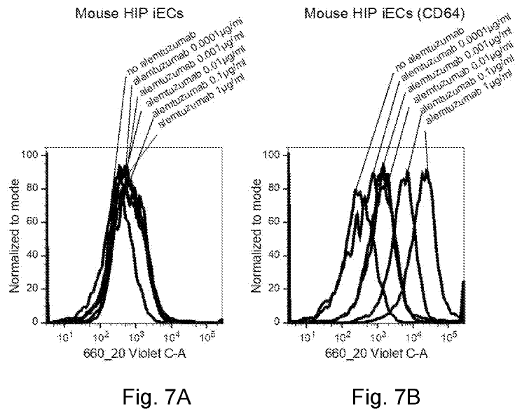

FIGS. 6A-6C show successful transfections with strong expression of the transgenes. Flow cytometry histograms of mouse HIP iECS (CD52, FIG. 6A), mouse HIP iECs (CD52 CD64, FIG. 6B), and mouse HIP iECs (CD64, FIG. 6C) are shown. Flow cytometry analysis was performed using antibodies against CD52 (CD52 APC: clone HI186, Biolegend) and CD64 (CD64 PE: clone 10.1, BD). All iEC pools were culture expanded for subsequent assays.

The ability of iECs to bind alemtuzumab Fc was assessed using flow cytometry. Cells were incubated with alemtuzumab (Cat #MCA6101, BioRad, Hercules, CA) at concentrations of 0.0001 μg/ml to 1.0 μg/ml. A goat anti-human IgG (H+L) F(ab′)2 secondary antibody (Cat #Q-11221MP, Qdot 655 tag, Invitrogen) was used to quantify alemtuzumab Fc binding. The analysis was done on an LSRFortessa cytometer (BD Biosciences).

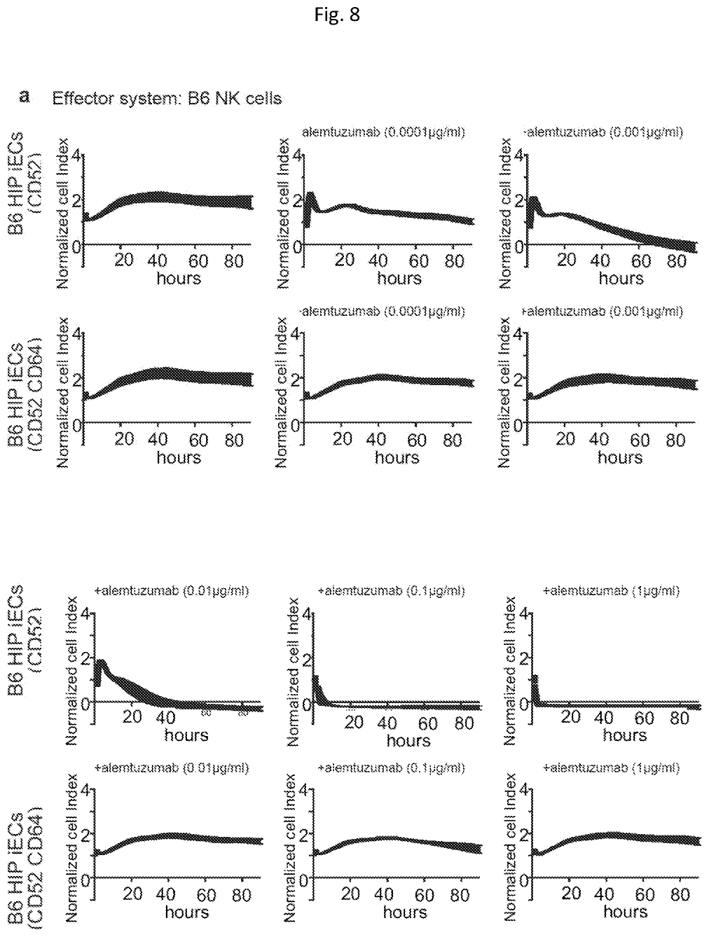

FIG. 7A shows that mouse HIP iECs did not bind any alemtuzumab. Mouse HIP iECs (CD64), however, were able to bind alemtuzumab Fc in a concentration-dependent manner (FIG. 7B).

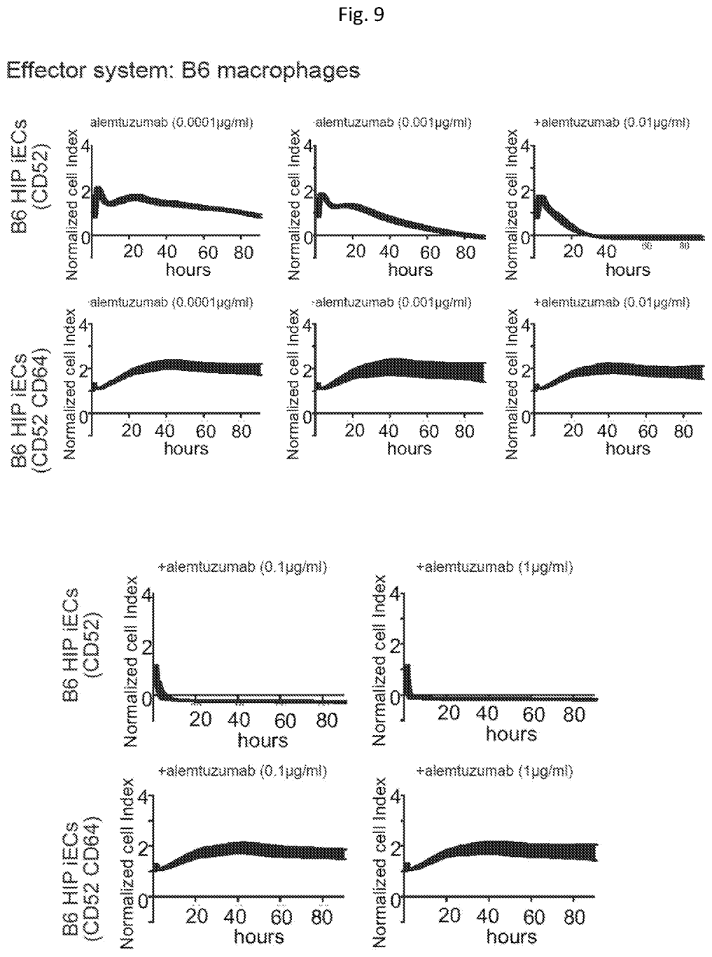

Mouse B6 iECs (CD52) and mouse B6 iECs (CD52, CD64) were assessed in XCelligence assays with different syngeneic B6 effector immune cells capable of ADCC. FIG. 8 upper row shows that the CD52+ iECs underwent concentration-dependent alemtuzumab-mediated NK cell ADCC. The CD64-expressing CD52 tg iECs in the lower row were protected against NK cell killing across all concentrations tested.

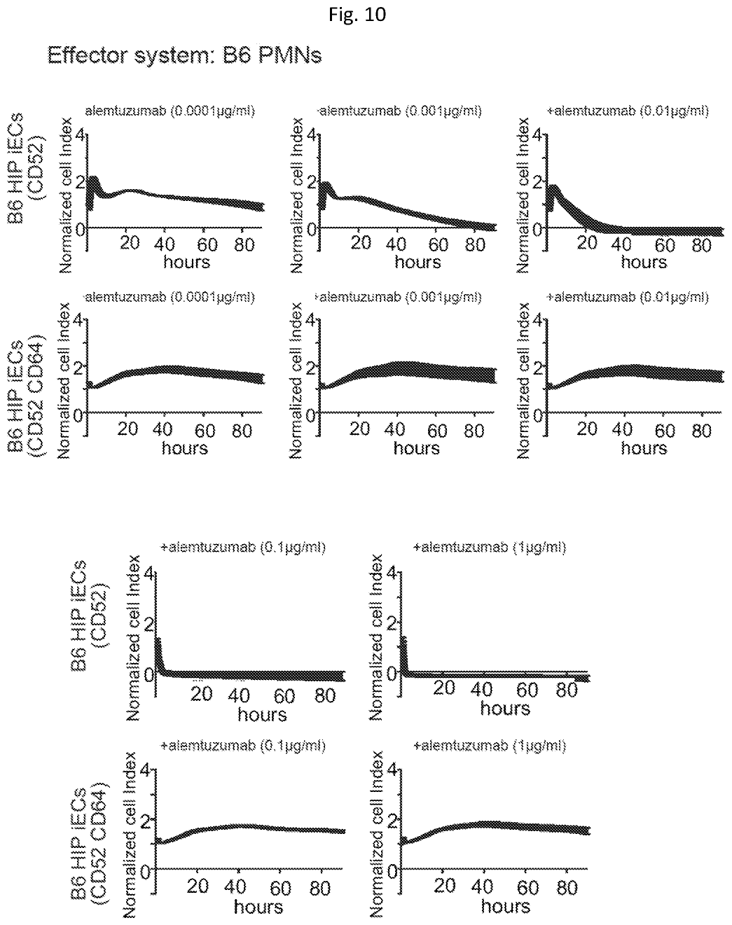

FIG. 9 shows the Xcelligence assays with syngeneic B6 macrophages as effector cells. The CD52+ iECs undergwent concentration-dependent alemtuzumab-mediated macrophage ADCC. The CD64-expressing CD52 tg iECs in the lower row were protected against macrophage killing across all concentrations tested.

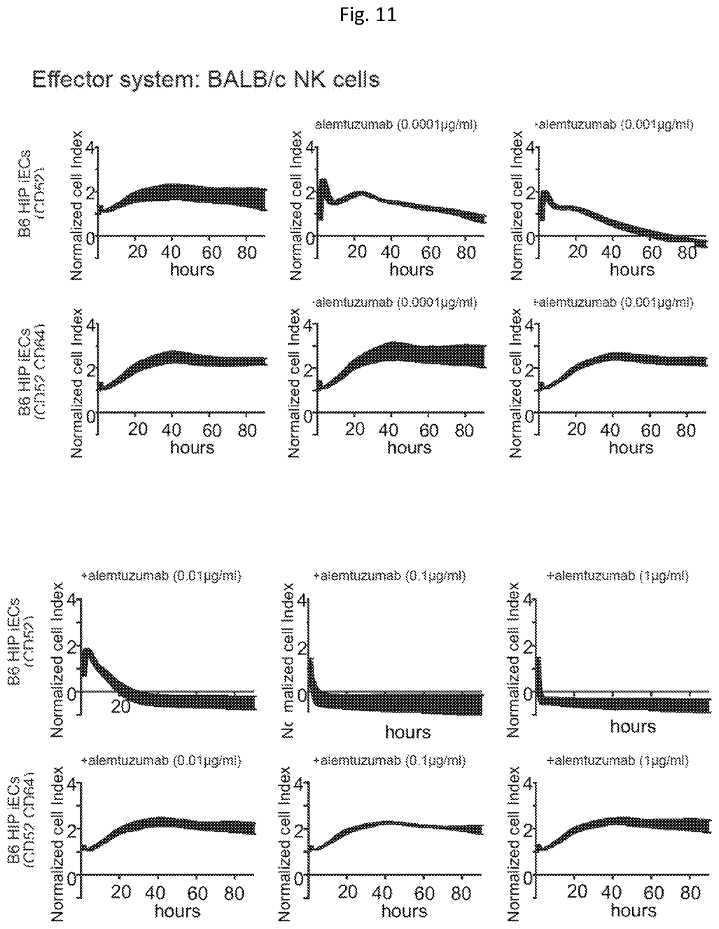

FIG. 10 shows the Xcelligence assays with syngeneic B6 polymorpho-nuclear cells (PMNs) as effector cells. The CD52+ iECs underwent concentration-dependent alemtuzumab-mediated PMN ADCC. CD64 co-expression in the lower row were protected the targets against PMN killing against across all concentrations tested. Thus, CD64 expression on iECs protected against all ADCC even at high alemtuzumab concentrations of 1.0 μg/ml.

To further confirm that CD64-expressing HIP cells remain protected from allogeneic NK cells, macrophages, and PMNs, the above analyses were repeated with allogeneic effector immune cells. In these assays, the effector cells were stimulated either by target cell antibody-binding or by allogeneic antigens detected via direct cell-cell interactions.

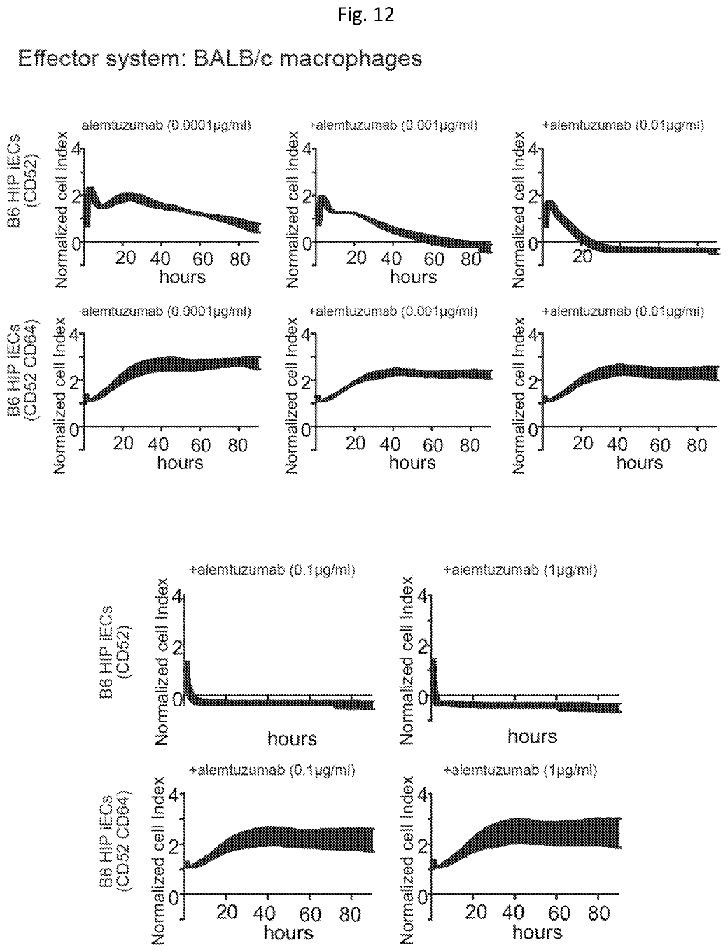

FIG. 11 upper row shows that the CD52+B6 HIP iECs underwent concentration-dependent alemtuzumab-mediated ADCC by allogeneic BALB/c NK cells.

The CD64-expressing CD52 tg HIP iECs in the lower row were protected against allogeneic NK cell killing across all concentrations tested. There was no additional direct killing by allogeneic NK cells.

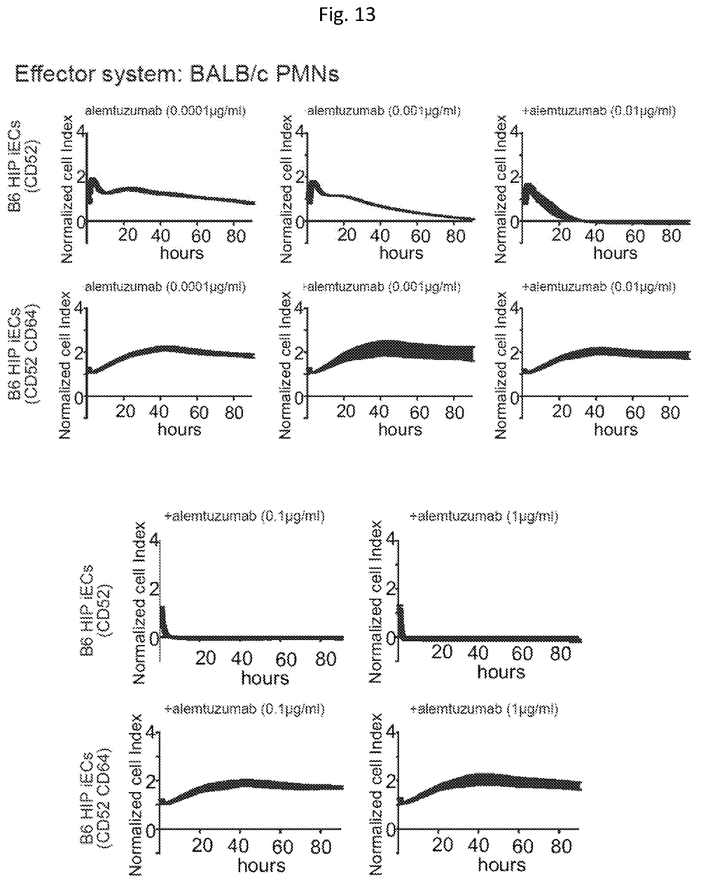

FIG. 12 shows the Xcelligence assays with allogeneic BALB/c macrophages as effector cells. The CD52+ HIP iECs underwent concentration-dependent alemtuzumab-mediated ADCC by allogeneic macrophages. The CD64-expressing CD52 tg HIP iECs in the lower row were protected against allogeneic macrophage killing across all concentrations tested. There was no additional direct killing by allogeneic macrophages.

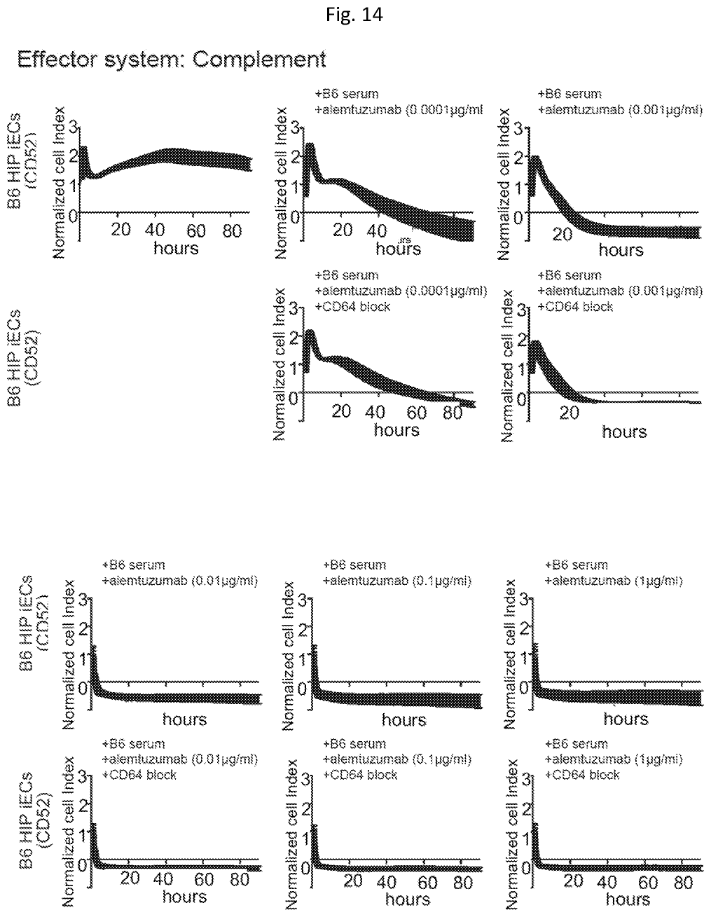

FIG. 13 shows the Xcelligence assays with allogeneic BALB/c PMNs as effector cells. The CD52+ HIP iECs underwent concentration-dependent alemtuzumab-mediated ADCC by allogeneic PMNs. CD64 co-expression in the lower row protected the targets against allogeneic PMN killing again across all concentrations tested. There was no additional direct killing by allogeneic PMNs. Thus, CD64 expressing HIP iECs were protected against syngeneic and allogeneic innate immune cell killing, both via ADCC and direct cytotoxicity.

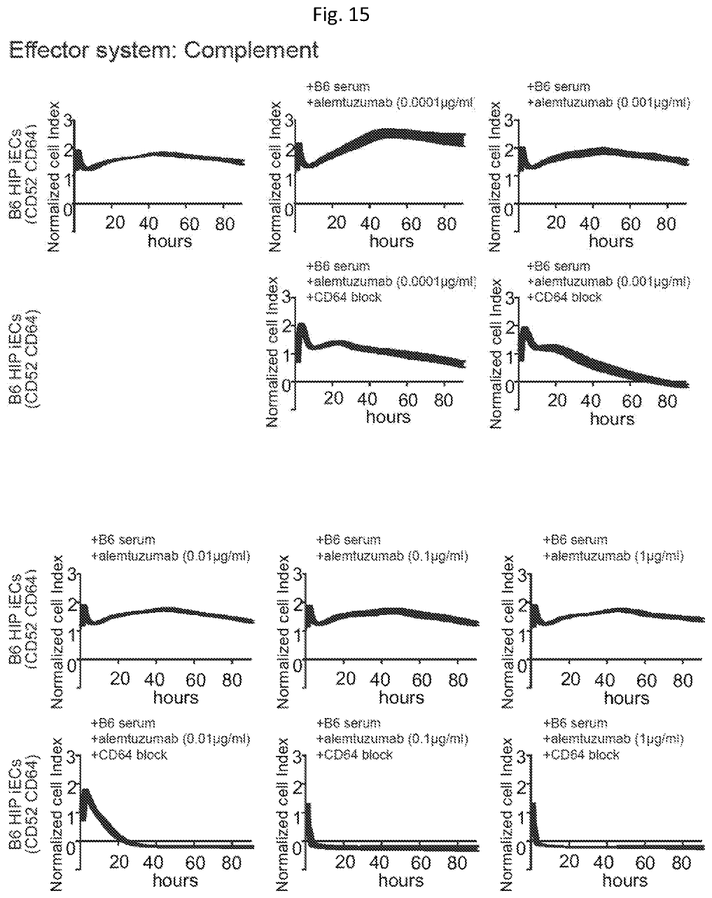

FIG. 14 shows mouse B6 HIP iECs (CD52) on the Xcelligence platform incubated with compatible, syngeneic B6 serum and increasing concentrations of alemtuzumab. CD52+ mouse B6 HIP iECs are susceptible to alemtuzumab-mediated CDC even at very low alemtuzumab concentrations of 0.0001 μg/ml (upper row). Since these targets do not express CD64, a blocking antibody against CD64 did not affect target cell killing (lower row).

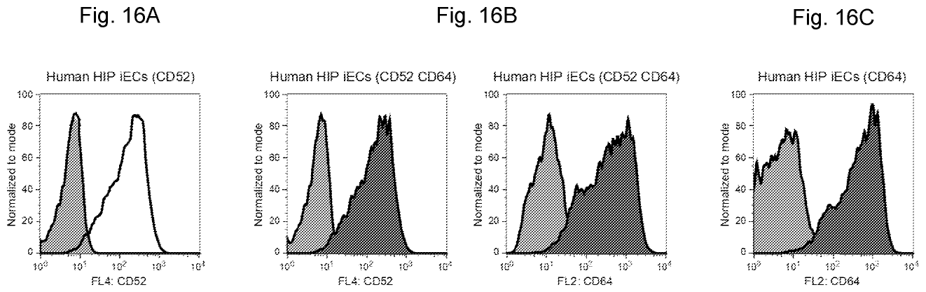

FIG. 15 shows that CD52+ mouse B6 HIP iECs co-expressing CD64 were protected against CDC across all alemtuzumab concentrations tested (upper row). A blocking antibody against CD64 abolished the protection and made the targets vulnerable to CDC (lower row).

### D. Example 4: CD64 Protected Engineered Human Endothelial Cells from ADCC and CDC Killing

Human HIP iPCS were differentiated into endothelial cells (iECs). The differentiation protocol was initiated at 60% HIP iPSC confluency, and medium was changed to RPMI-1640 containing 2% B-27 minus insulin (both Gibco, a Thermo Fisher Scientific Brand) and 5 μM CHIR-99021 (Selleckchem, Munich, Germany). On day 2, the medium was changed to reduced medium: RPMI-1640 containing 2% B-27 minus insulin (Gibco) and 2 μM CHIR-99021 (Selleckchem). From culture day 4 to 7, cells were exposed to RPMI-1640 EC medium, RPMI-1640 containing 2% B-27 minus insulin plus 50 ng/ml human vascular endothelial growth factor (VEGF; R&D Systems, Minneapolis, MN), 10 ng/ml human fibroblast growth factor basic (FGFb; R&D Systems), 10 μM Y-27632 (Sigma-Aldrich), and 1 μM SB 431542 (Sigma-Aldrich, St. Louis, MO). Endothelial cell clusters were visible from day 7 and cells were maintained in Endothelial Cell Basal Medium 2 (PromoCell, Heidelberg, Germany) plus supplements, 10% FCS hi (Gibco), 1% pen/strep, 25 ng/ml VEGF, 2 ng/ml FGFb, 10 μM Y-27632 (Sigma-Aldrich), and 1 μM SB 431542 (Sigma-Aldrich). The differentiation protocol was completed after 14 days; undifferentiated cells detached during the differentiation process. TrypLE Express (Gibco) was used for passaging the cells 1:3 every 3 to 4 days.

Some human HIP iECs were then transformed with a lentiviral vector that expresses CD52: For transfection analyses, 1.5×10⁵ human HIP iECs were plated per well of a 6-well plate. The cells were incubated overnight at 37° C. in a cell incubator. The next day, transfection was performed using Fugene (Promaga, Fitchburg, WI) and 5 μg of CD52 expression particles (Origene, Rockville, MD, CAT #: RC200493L2V) in a ratio of 3:2. The transfection reagent solution was pipetted to OptiMEM (Gibco), mixed, and incubated for 10 minutes at room temperature. The DNA transfection complex was added to 2 ml of cell medium. After 24 h, the transfection was stopped, and cells were grown for further 48 h in endothelial cell medium (Gibco).

A subset of the human HIP iECs (CD52) were further transfected with a lentivirus to express CD64 using the same protocol. The transfection was performed using Fugene (Promaga) and 5 μg of CD64 expression plasmids (Origene, Rockville, MD, CAT #: RC207487L2V) in a ratio of 3:2. The transfection was stopped as outlined above and cells were expanded for another 48 h.

Some HIP-derived human iECs were transduced to only express CD64 according to the protocol above.

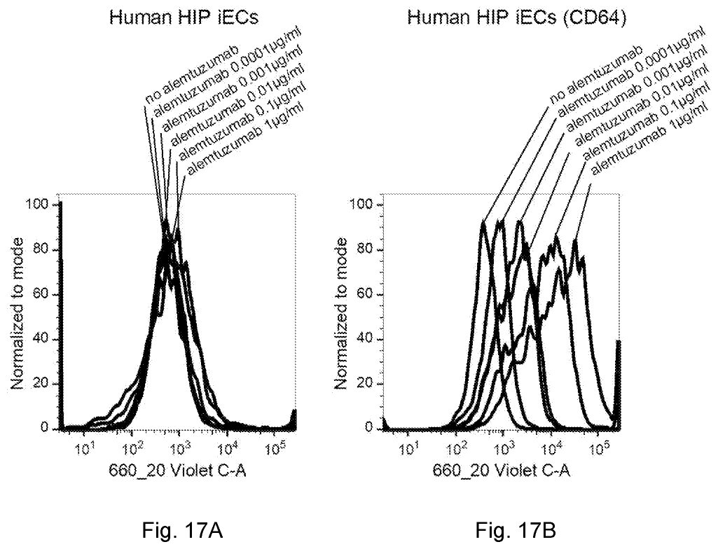

FIGS. 16A-16C show successful transfections with strong expression of the transgenes. Flow cytometry histograms of human HIP iECs (CD52, FIG. 16A), human HIP iECs (CD52 CD64, FIG. 16B), and mouse HIP iECs (CD64, FIG. 16C) are shown. Flow cytometry analysis was performed using antibodies against CD52 (CD52 APC: clone HI186, Biolegend) and CD64 (CD64 PE: clone 10.1, BD). All iEC pools were culture expanded for subsequent assays.

The ability of human HIP iECs to bind alemtuzumab Fc was assessed using flow cytometry. Cells were incubated with alemtuzumab (Cat #MCA6101, BioRad, Hercules, CA) at concentrations of 0.0001 μg/ml to 1.0 μg/ml. A goat anti-human IgG (H+L) F(ab′)2 secondary antibody (Cat #Q-11221MP, Qdot 655 tag, Invitrogen) was used to quantify alemtuzumab Fc binding. The analysis was done on an LSRFortessa cytometer (BD Biosciences).

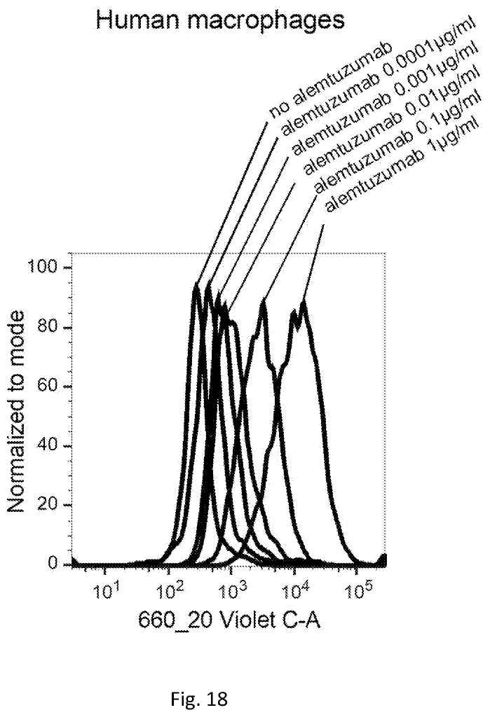

FIG. 17A shows that human HIP iECs did not bind any alemtuzumab. Human HIP iECs (CD64), however, were able to bind alemtuzumab Fc in a concentration-dependent manner (FIG. 17B).

Since macrophages constitutively express CD64, they were used as a control to compare the alemtuzumab Fc binding capacity of human HIP iECs. PBMCs were isolated by Ficoll separation from fresh volunteer blood and were resuspended in RPMI 1640 with 10% FCS hi, 1% pen-strep (all Gibco) and 10 ng/ml human M-CSF (Peprotech). Cells were plated in 24-well plates at a concentration of 1×10⁶ cells per ml (1 ml per 24 well plate) and medium was changed every second day until day 6. Macrophages were stimulated from day 6 with and 1p g/ml human IL2 (Peprotech) for 24 hours before the cells were used for assays.

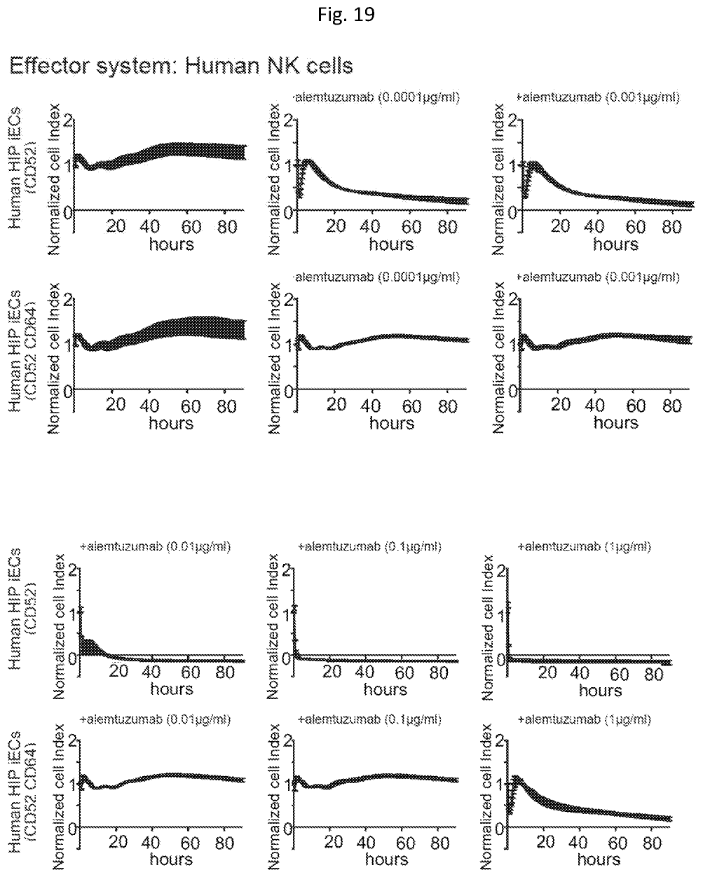

FIG. 18 shows the ability of human macrophages to bind alemtuzumab Fc. Alemtuzumab Fc binding was concentration-dependent.

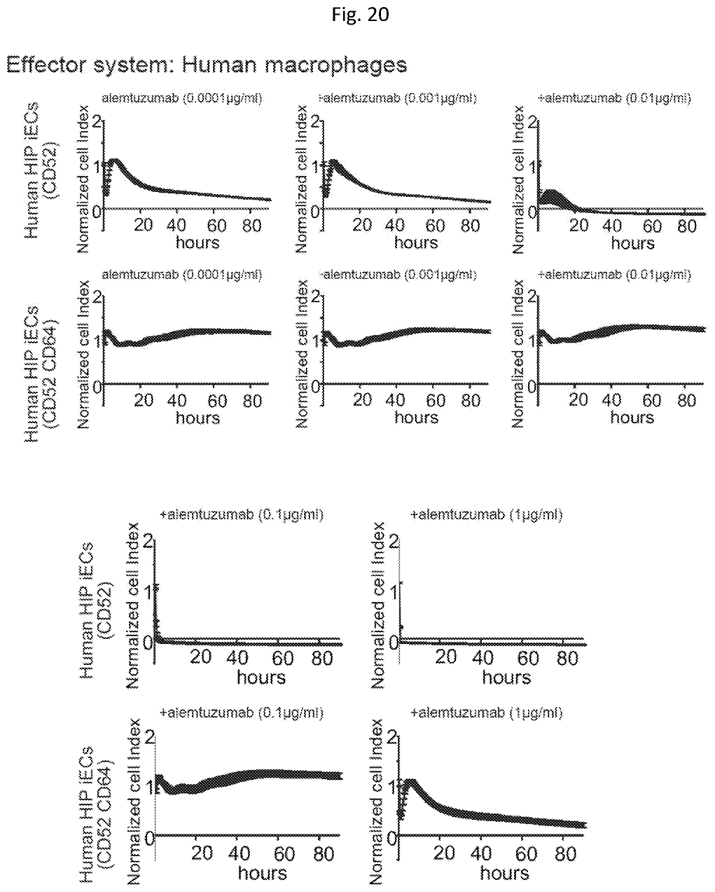

FIG. 19 upper row shows that CD52+ human HIP iECs undergo concentration-dependent alemtuzumab-mediated ADCC by allogeneic NK cells. The CD64-expressing CD52 tg HIP iECs in the lower row were largely protected against allogeneic NK cell killing. Killing occurred only at 1.0 μg/ml of the anti-CD52 antibody (alemtuzumab). There was no additional direct killing by allogeneic NK cells.

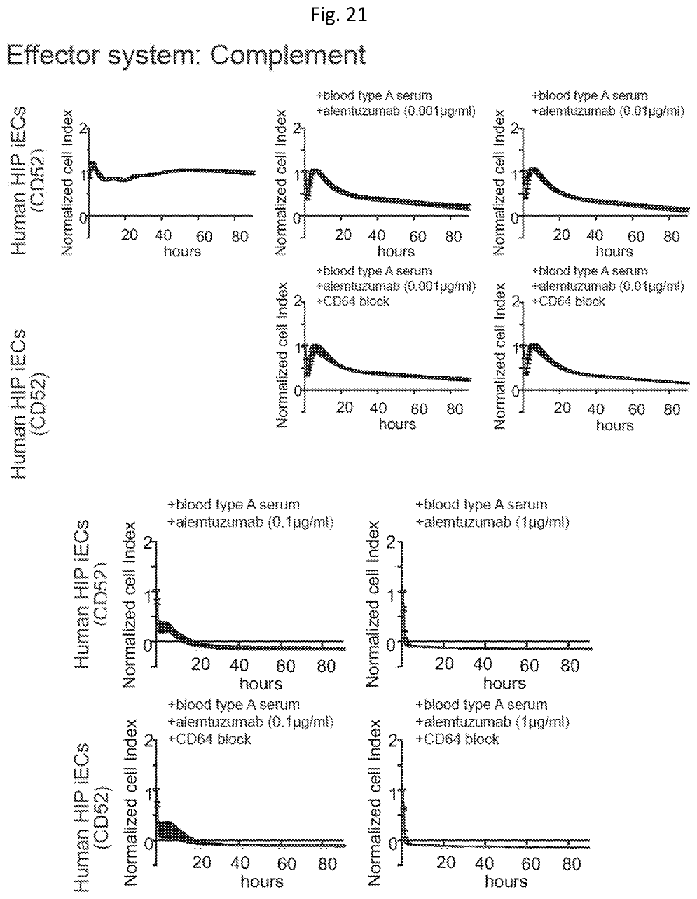

FIG. 20 upper row shows that CD52+ human HIP iECs underwent concentration-dependent alemtuzumab-mediated ADCC by allogeneic macrophages. The CD64-expressing CD52 tg HIP iECs in the lower row were largely protected against allogeneic NK cell killing. Killing occurred only at 1.0 μg/ml of the anti-CD52 antibody (alemtuzumab). There was no additional direct killing by allogeneic macrophages. Thus, CD64 expressing human HIP iECs were protected against allogeneic NK cell and macrophage killing, both via ADCC and direct cytotoxicity.

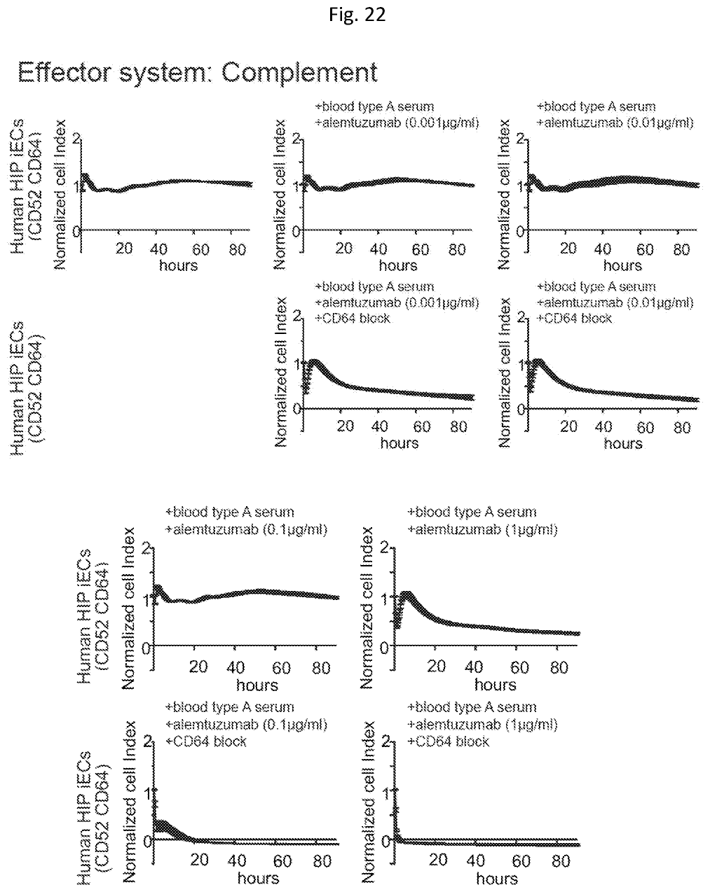

FIG. 21 shows human HIP iECs (CD52) on the XCelligence platform incubated with compatible, ABO-matched allogeneic serum and increasing concentrations of alemtuzumab. CD52+ human HIP iECs were susceptible to alemtuzumab-mediated CDC even at very low alemtuzumab concentrations of 0.001 μg/ml (upper row). Since these targets did not express CD64, a blocking antibody against CD64 did not affect target cell killing (lower row).

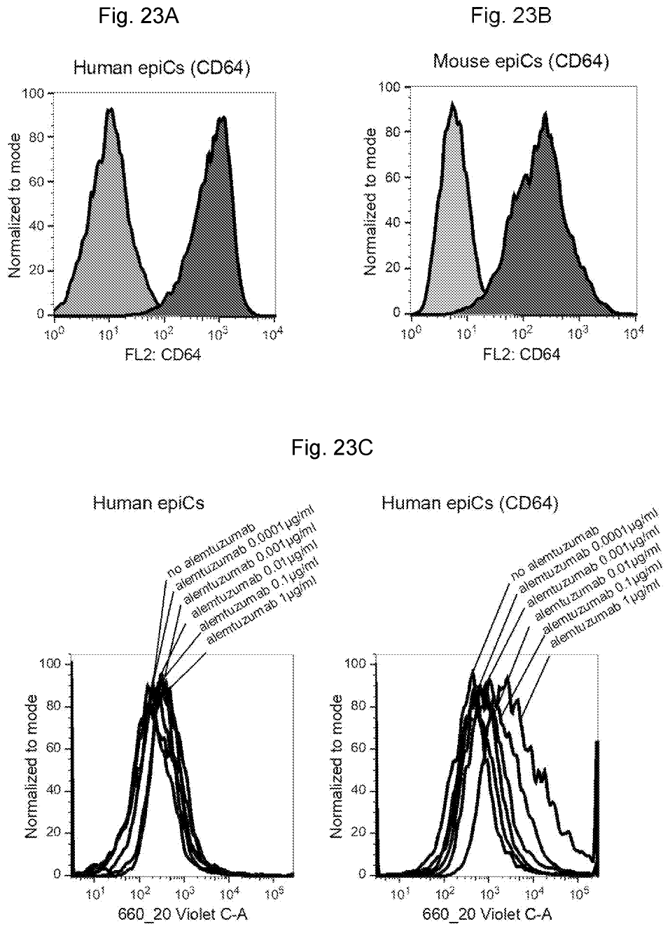

FIG. 22 shows that CD52+ human HIP iECs co-expressing CD64 were largely protected against CDC (upper row). Killing occurred only at 1.0 μg/ml of the anti-CD52 antibody (alemtuzumab). A blocking antibody against CD64 abolished the protection and made the targets vulnerable to CDC (lower row).

### E. Example 5: CD64 Expression on Thyroid Epithelial Cells Sequestered IgG Antibody Fc

CD64 expression on target cells to sequester IgG Fc in order to protect the cells from ADCC and CDC was shown in thyroid epithelial cells. These cells are typically attacked by autoimmune antibodies in Hashimoto disease and thus a clinically-relevant cell type. Mouse B6 thyroid epithelial cells (Cat no: mGFP-6040, Cell Biologics, Chicago, IL) and immortalized human thyroid epithelial cells (Cat no: INS-CI-1017, InSCREENeX, Braunschweig, Germany) were purchased. CD64 expression was achieved as outlined above.

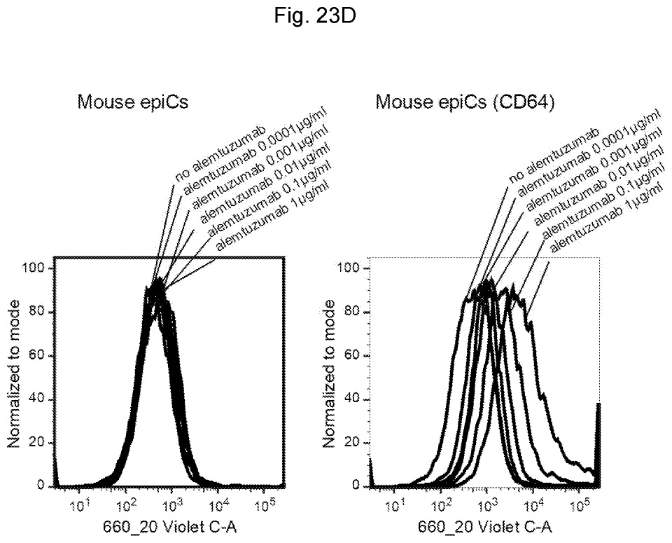

FIG. 23 show the successful expression of CD64 in human thyroid epithelial cells (epiCs, FIG. 23A) and mouse epiCs (FIG. 23B). The ability of the cells to bind alemtuzumab Fc was assessed in binding assays as described above. Human epiCs were unable to bind alemtuzumab Fc, but CD64+ human epiCs showed a concentration-dependent binding of IgG Fc (FIG. 23C). Similarly, mouse epiCs were unable to bind alemtuzumab Fc, but CD64+ mouse epiCs showed a concentration-dependent binding of IgG Fc (FIG. 23D).

Next, the sequestration of anti-TPO Fc by CD64 expressing human and mouse epiCs was analyzed. Anti-TPO antibodies induced in autoimmune thyroiditis (Hashimoto's disease) are responsible for the destruction of thyroid cells in humans and for the establishment of hypothyroidism. A disease model was designed that used a rabbit-anti-mouse TPO antibody (polyclonal, Cat no: ab203057, Abcam, Cambridge, MA). This antibody recognizes TPO on mouse thyroid epithelial cells and can bind to human CD64 via Fc.

The ability of human and mouse thyroid epiCs to bind anti-TPO Fc was assessed using flow cytometry. Cells were incubated with anti-TPO (polyclonal, Cat no: ab203057, Abcam, Cambridge, MA) at concentrations of 0.0001 μg/ml to 1.0 μg/ml. A goat anti-rabbit IgG (H+L) F(ab′)2 secondary antibody (Cat #Q11422MP, Qdot 655 tag, Invitrogen) was used to quantify anti-TPO Fc binding. The analysis was done on an LSRFortessa cytometer (BD Biosciences).

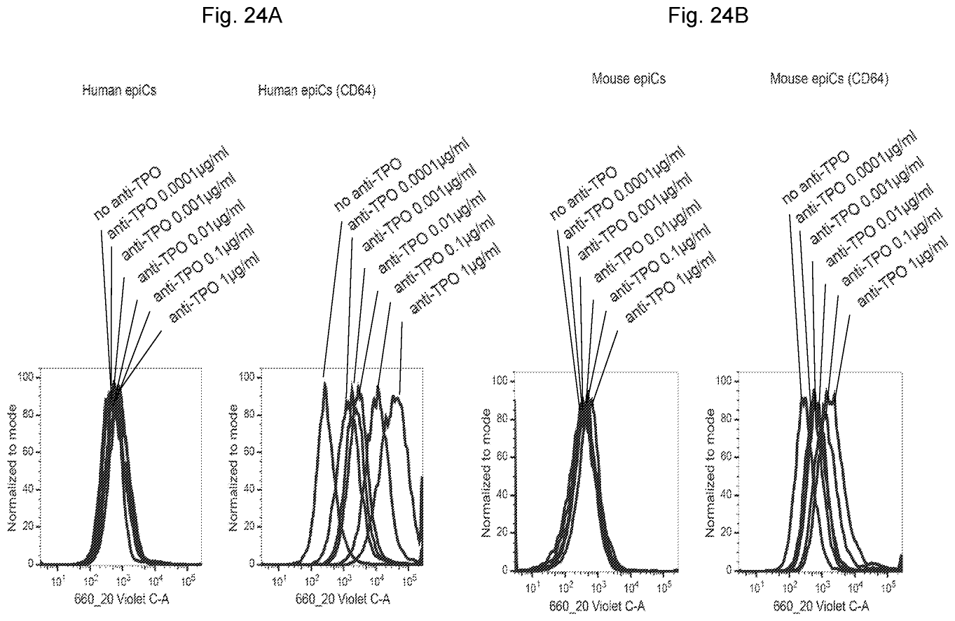

FIGS. 24A and B show that human epiCs were unable to bind anti-TPO Fc, but CD64+ human epiCs showed a concentration-dependent binding of anti-TPO Fc (FIG. 24A). Similarly, mouse epiCs were unable to bind anti-TPO Fc, but CD64+ mouse epiCs showed a concentration-dependent binding of anti-TPO Fc (FIG. 23B). The concept of Fc sequestration was thus confirmed in a clinically-relevant cell type.

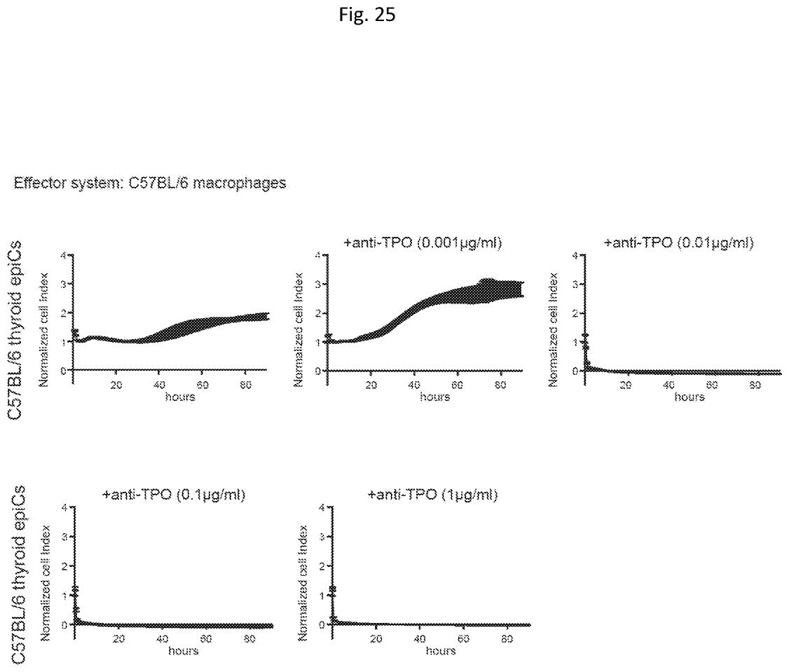

FIG. 25 shows the in vitro killing of C57BL/6 thyroid epiCs on the XCelligence platform. Target cells were incubated with different concentrations of anti-TPO (0.001 μg/ml to 1.0 μg/ml) and with syngeneic macrophages as effector cells for ADCC. Target cell killing was observed at anti-TPO concentrations of 0.01 μg/ml to 1.0 μg/ml. This showed that mouse epiCs were susceptible to anti-TPO ADCC in vitro.

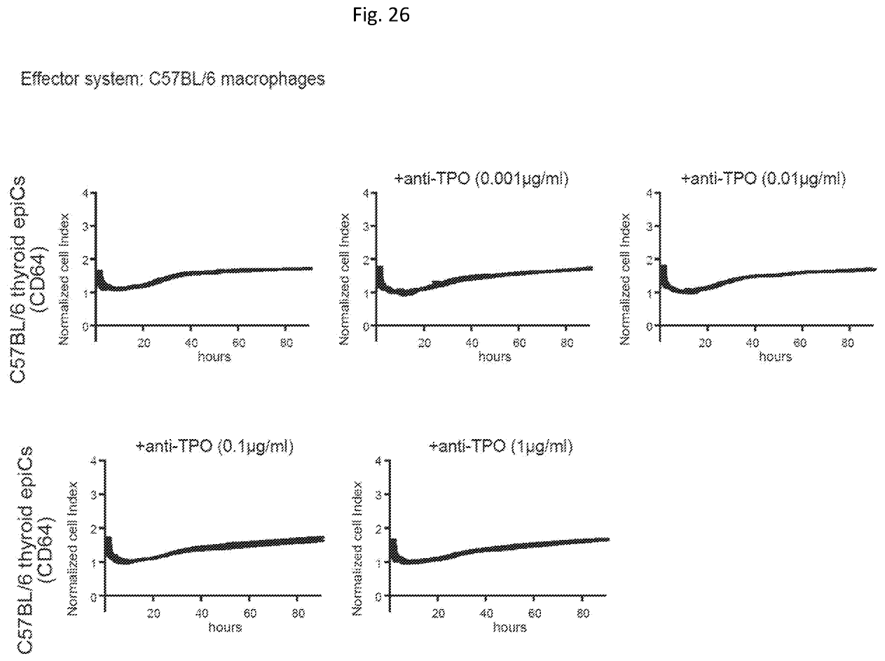

FIG. 26 shows the same analytical design but used C57BL/6 thyroid epiCs expressing CD64. When incubated with anti-TPO and syngeneic macrophages, no killing was observed in any of the anti-TPO concentrations. CD64 expression proved effective to protect mouse thyroid epiCs from anti-TPO ADCC.

### F. Example 6: CD64 Expression Protected Mouse Endothelial Cells from Killing In Vivo

An in vivo model showed that CD64 protected mouse endothelial cells from antibody-mediated rejection. Syngeneic C57BL/6 mice were chosen as recipients of C57BL/6 HIP iECs to avoid allogeneic cellular rejection, and thus, the model was purely antibody-driven. One million CD52-expressing mouse HIP iECs were transplanted subcutaneously.

Alemtuzumab was administered at 1 mg doses on days 0 and 3 after transplantation intraperitoneally. The animals were imaged daily using BLI to assess graft cell survival.

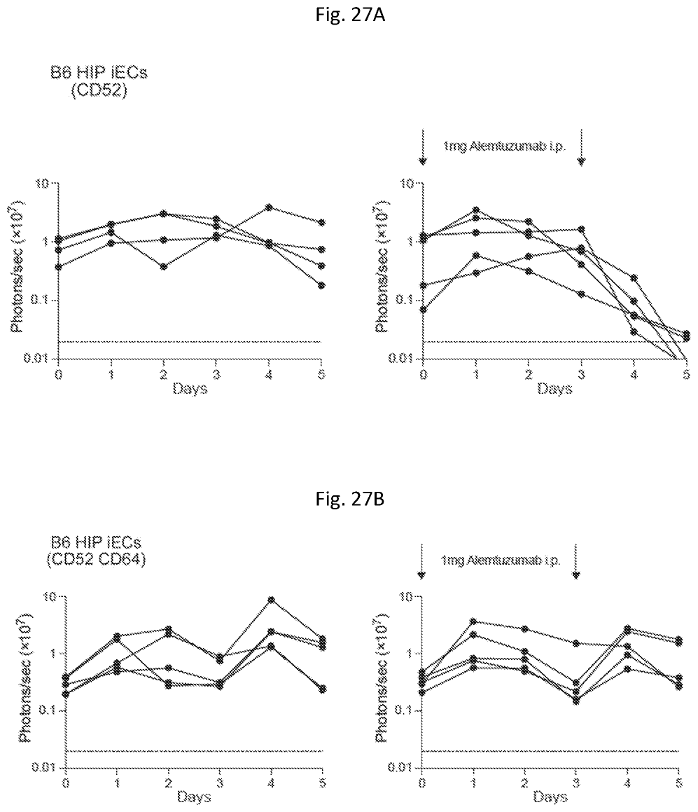

FIGS. 27A and 27B show the graft survival in vivo. FIG. 27A shows the survival of C57BL/6 HIP iECs (CD52) without alemtuzumab (left) and with alemtuzumab treatment (right). Alemtuzumab administration resulted in 100% graft loss on day 5. FIG. 27B shows the survival of C57BL/6 HIP iECs (CD52, CD64) without alemtuzumab (left) and with alemtuzumab treatment (right). All grafts were protected from antibody-mediated rejection and all grafts survived the study period without relevant drops in BLI signal. This analysis confirmed that CD64 expression makes target cells resistant against antibody-mediated rejection. This in vivo model combined the ADCC and CDC killing mechanisms.

### IX. Exemplary Sequences

Human β-2-Microglobulin
SEQ ID NO: 1
MSRSVALAVLALLSLSGLEAIQRTPKIQVYSRHPAENGKSNFLNC
YVSGFHPSDIEVDLLKNGERIEKVEHSDLSFSKDWSFYLLYYTEF
TPTEKDEYACRVNHVTLSQPKIVKWDRDI
Human CIITA protein, 160 amino acid N-terminus
SEQ ID NO: 2
MRCLAPRPAGSYLSEPQGSSQCATMELGPLEGGYLELLNSDADPL
CLYHFYDQMDLAGEEEIELYSEPDTDTINCDQFSRLLCDMEGDEE
TREAYANIAELDQYVFQDSQLEGLSKDIFKHIGPDEVIGESMEMP
AEVGQKSQKRPFPEELPADLKHWKP
Human CD47
SEQ ID NO: 3
MWPLVAALLLGSACCGSAQLLENKTKSVEFTFCNDTVVIPCFVTN
MEAQNTTEVYVKWKFKGRDIYTFDGALNKSTVPTDESSAKIEVSQ
LLKGDASLKMDKSDAVSHTGNYTCEVTELTREGETIIELKYRVVS
WFSPNENILIVIFPIFAILLFWGQFGIKTLKYRSGGMDEKTIALL
VAGLVITVIVIVGAILFVPGEYSLKNATGLGLIVTSTGILILLHY
YVESTAIGLTSFVIAILVIQVIAYILAVVGLSLCIAACIPMHGPL
LISGLSILALAQLLGLVYMKEVE
Herpes Simplex Virus Thimidine Kinase (HSV-tk)
SEQ ID NO: 4
MASYPCHQHASAFDQAARSRGHSNRRTALRPRRQQEATEVRLEQK
MPTLLRVYIDGPHGMGKTTTTQLLVALGSRDDIVYVPEPMTYWQV
LGASETIANIYTTQHRLDQGEISAGDAAVVMTSAQITMGMPYAVT
DAVLAPHVGGEAGSSHAPPPALTLIFDRHPIAALLCYPAARYLMG
SMTPQAVLAFVALIPPTLPGTNIVLGALPEDRHIDRLAKRQRPGE
RLDLAMLAAIRRVYGLLANTVRYLQGGGSWWEDWGQLSGTAVPPQ
GAEPQSNAGPRPHIGDTLFTLFRAPELLAPNGDLYNVFAWALDVL
AKRLRPMHVFILDYDQSPAGCRDALLQLTSGMVQTHVTTPGSIPT
ICDLARTFAREMGEAN
Escherichia coli Cytosine Deaminase (EC-CD)
SEQ ID NO: 5
MSNNALQTIINARLPGEEGLWQIHLQDGKISAIDAQSGVMPITEN
SLDAEQGLVIPPFVEPHIHLDTTQTAGQPNWNQSGTLFEGIERWA
ERKALLTHDDVKQRAWQTLKWQIANGIQHVRTHVDVSDATLTALK
AMLEVKQEVAPWIDLQIVAFPQEGILSYPNGEALLEEALRLGADV
VGAIPHFEFTREYGVESLHKTFALAQKYDRLIDVHCDEIDDEQSR
EVETVAALAHHEGMGARVTASHTTAMHSYNGAYTSRLFRLLKMSG
INFVANPLVNIHLQGREDTYPKRRGITRVKEMLESGINVCFGHDD
VFDPWYPLGTANMLQVLHMGLHVCQLMGYGQINDGLNLITHHSAR
TLNLQDYGIAAGNSANLIILPAENGFDALRRQVPVRYSVRGGKVI
ASTQPAQTTVYLEQPEAIDYKR
Truncated human Caspase 9
SEQ ID NO: 6
GFGDVGALESLRGNADLAYILSMEPCGHCLIINNVNFCRESGLRT
RTGSNIDCEKLRRRESSLHFMVEVKGDLTAKKMVLALLELAQQDH
GALDCCVVVILSHGCQASHLQFPGAVYGTDGCPVSVEKIVNIFNG
TSCPSLGGKPKLFFIQACGGEQKDHGFEVASTSPEDESPGSNPEP
DATPFQEGLRTFDQLDAISSLPTPSDIFVSYSTFPGFVSWRDPKS
GSWYVETLDDIFEQWAHSEDLQSLLLRVANAVSVKGIYKQMPGCF
NFLRKKLFFKTS
Human CD64
NM_000566
SEQ ID NO: 7
MWFLTTLLLWVPVDGQVDTTKAVITLQPPWVSVFQEETVTLHCEVL
HLPGSSSTQWFLNGTATQTSTPSYRITSASVNDSGEYRCQRGLSG
RSDPIQLEIHRGWLLLQVSSRVFTEGEPLALRCHAWKDKLVYNVL
YYRNGKAFKFFHWNSNLTILKTNISHNGTYHCSGMGKHRYTSAGI
SVTVKELFPAPVLNASVTSPLLEGNLVTLSCETKLLLQRPGLQLY
FSFYMGSKTLRGRNTSSEYQILTARREDSGLYWCEAATEDGNVLK
RSPELELQVLGLQLPTPVWFHVLFYLAVGIMFLVNTVLWVTIRKE
LKRKKKWDLEISLDSGHEKKVISSLQEDRHLEEELKCQEQKEEQL
QEGVHRKEPQGAT
Human CD52
NM_001803
SEQ ID NO: 8
MKRFLFLLLTISLLVMVQIQTGLSGQNDTSQTSSPSASSNISGGI
FLFFVANAIIHLFCES
Human CD16 FCGR3A
NM_001803
SEQ ID NO: 9
MAEGTLWQILCVSSDAQPQTFEGVKGADPPTLPPGSFLPGPVLWW
GSLARLQTEKSDEVSRKGNWWVTEMGGGAGERLFTSSCLVGLVPL
GLRISLVTCPLQCGIMWQLLLPTALLLLVSAGMRTEDLPKAVVFL
EPQWYRVLEKDSVTLKCQGAYSPEDNSTQWFHNESLISSQASSYF
IDAATVDDSGEYRCQTNLSTLSDPVQLEVHIGWLLLQAPRWVEKE
EDPIHLRCHSWKNTALHKVTYLQNGKGRKYFHHNSDFYIPKATLK
DSGSYFCRGLFGSKNVSSETVNITITQGLAVSTISSFFPPGYQVS
FCLVMVLLFAVDTGLYFSVKTNIRSSTRDWKDHKFKWRKDPQDK
Human CD16 FCGR3B
NM_001803
SEQ ID NO: 10
MWQLLLPTALLLLVSAGMRTEDLPKAVVFLEPQWYSVLEKDSVTL
KCQGAYSPEDNSTQWFHNENLISSQASSYFIDAATVNDSGEYRCQ
TNLSTLSDPVQLEVHIGWLLLQAPRWVFKEEDPIHLRCHSWKNTA
LHKVTYLQNGKDRKYFHHNSDFHIPKATLKDSGSYFCRGLVGSKN
VSSETVNITITQGLAVSTISSFSPPGYQVSFCLVMVLLFAVDTGL
YFSVKTNI
Human CD32 FCGR2A
NM_001803
SEQ ID NO: 11
MTMETQMSQNVCPRNLWLLQPLTVLLLLASADSQAAAPPKAVLKL
EPPWINVLQEDSVTLTCQGARSPESDSIQWFHNGNLIPTHTQPSY
RFKANNNDSGEYTCQTGQTSLSDPVHLTVLSEWLVLQTPHLEFQE
GETIMLRCHSWKDKPLVKVTFFQNGKSQKFSHLDPTFSIPQANHS
HSGDYHCTGNIGYTLFSSKPVTITVQVPSMGSSSPMGIIVAVVIA
TAVAAIVAAVVALIYCRKKRISANSTDPVKAAQFEPPGRQMIAIR
KRQLEETNNDYETADGGYMTLNPRAPTDDDKNIYLTLPPNDHVNS
NN
Human CD32 FCGR2B
NM_001803
SEQ ID NO: 12
MGILSFLPVLATESDWADCKSPQPWGHMLLWTAVLFLAPVAGTPA
APPKAVLKLEPQWINVLQEDSVTLTCRGTHSPESDSIQWFHNGNL
IPTHTQPSYRFKANNNDSGEYTCQTGQTSLSDPVHLTVLSEWLVL
QTPHLEFQEGETIVLRCHSWKDKPLVKVTFFQNGKSKKESRSDPN
ESIPQANHSHSGDYHCTGNIGYTLYSSKPVTITVQAPSSSPMGII
VAVVTGIAVAAIVAAVVALIYCRKKRISALPGYPECREMGETLPE
KPANPTNPDEADKVGAENTITYSLLMHPDALEEPDDQNRI
Human CD32 FCGR2C
NM_001803
SEQ ID NO: 13
MGILSFLPVLATESDWADCKSPQPWGHMLLWTAVLFLAPVAGTPA
APPKAVLKLEPQWINVLQEDSVTLTCRGTHSPESDSIPWFHNGNL
IPTHTQPSYRFKANNNDSGEYTCQTGQTSLSDPVHLTVLSEWLVL
QTPHLEFQEGETIVLRCHSWKDKPLVKVTFFQNGKSKKFSRSDPN
ESIPQANHSHSGDYHCTGNIGYTLYSSKPVTITVQAPSSSPMGII
VAVVTGIAVAAIVAAVVALIYCRKKRISANSTDPVKAAQFEPPGR
QMIAIRKRQPEETNNDYETADGGYMTLNPRAPTDDDKNIYLTLPP
NDHVNSNN

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1. A modified pluripotent cell, wherein said modified pluripotent cell has an elevated level of CD16, CD32, or CD64 protein expression when compared to a parental version of said modified pluripotent cell, wherein said elevated protein expression causes said modified pluripotent cell to be less susceptible to antibody dependent cellular cytoxicity (ADCC) or complement-dependent cytotoxicity (CDC).

2. The modified pluripotent cell of claim 1, wherein said elevated protein expression is a CD64 protein, and wherein said CD64 protein has at least a 90% sequence identity to SEQ ID NO:7.

3. The modified pluripotent cell of claim 2, wherein said CD64 protein has the sequence of SEQ ID NO:7

4. The modified pluripotent cell of claim 1, wherein said modified cell is derived from a human hypo-immunogenic pluripotent (HIP) cell.

5. The modified pluripotent cell of claim 1, wherein said modified cell is derived from a human hypo-immunogenic pluripotent ABO blood group O Rhesus Factor negative (HIPO−) cell.

6. The modified pluripotent cell of claim 1, wherein said modified cell is derived from a human induced pluripotent stem cell (iPSC).

7. The modified pluripotent cell of claim 1, wherein said modified cell is derived from a human embryonic stem cell (ESC).

8. The modified pluripotent cell of claim 1, wherein said modified cell is from a species that is selected from the group consisting of a human, monkey, cow, pig, chicken, turkey, horse, sheep, goat, donkey, mule, duck, goose, buffalo, camel, yak, llama, alpaca, mouse, rat, dog, cat, hamster, and guinea pig.

9. The modified pluripotent cell of any one of claims 1-8, further comprising a suicide gene that is activated by a trigger that causes said modified cell to die.

10. The modified pluripotent cell of claim 9, wherein said suicide gene is a herpes simplex virus thymidine kinase gene (HSV-tk) and said trigger is ganciclovir.

11. The modified pluripotent cell of claim 10, wherein said HSV-tk gene encodes a protein comprising at least a 90% sequence identity to SEQ ID NO:4.

12. The modified pluripotent cell of claim 11, wherein said HSV-tk gene encodes a protein comprising the sequence of SEQ ID NO:4.

13. The modified pluripotent cell of claim 9, wherein said suicide gene is an Escherichia coli cytosine deaminase gene (EC-CD) and said trigger is 5-fluorocytosine (5-FC).

14. The modified pluripotent cell of claim 13, wherein said EC-CD gene encodes a protein comprising at least a 90% sequence identity to SEQ ID NO:5.

15. The modified pluripotent cell of claim 14, wherein said EC-CD gene encodes a protein comprising the sequence of SEQ ID NO:5.

16. The modified pluripotent cell of claim 9, wherein said suicide gene encodes an inducible Caspase protein and said trigger is a chemical inducer of dimerization (CID).

17. The modified pluripotent cell of claim 16, wherein said gene encodes an inducible Caspase protein comprising at least a 90% sequence identity to SEQ ID NO:6.

18. The modified pluripotent cell of claim 17, wherein said gene encodes an inducible Caspase protein comprising the sequence of SEQ ID NO:6.

19. The modified pluripotent cell of any one of claims 16-18, wherein said CID is AP1903.

20. A cell derived from the modified pluripotent cell of any one of claims 1-19, wherein said derivative cell is selected from the group consisting of a chimeric antigen receptor (CAR) cell, an endothelial cell, a dopaminergic neuron, a pancreatic islet cell, a cardiomyocyte, a retinal pigment endothelium cell, and a thyroid cell.

21. The modified pluripotent cell of claim 20, wherein said CAR cell is a CAR-T cell.

22. A method, comprising transplanting a cell derived from said modified pluripotent cell of any one of claims 1-19 into a subject, wherein said subject is a human, monkey, cow, pig, chicken, turkey, horse, sheep, goat, donkey, mule, duck, goose, buffalo, camel, yak, llama, alpaca, mouse, rat, dog, cat, hamster, guinea pig.

23. The method of claim 22, wherein said cell derived from said modified pluripotent cell is selected from the group consisting of a chimeric antigen receptor (CAR) cell, an endothelial cell, a dopaminergic neuron, a pancreatic islet cell, a cardiomyocyte, and a retinal pigment endothelium cell.

24. A method of treating a disease, comprising administering a cell derived from the modified pluripotent cell of any one of claims 1-19.

25. The method of claim 24, wherein said derivative cell is selected from the group consisting of a chimeric antigen receptor (CAR) cell, an endothelial cell, a dopaminergic neuron, a pancreatic islet cell, a cardiomyocyte, a retinal pigment endothelium cell and a thyroid cell.

26. The method of claim 24, wherein said disease is selected from the group consisting of Type I Diabetes, a cardiac disease, a neurological disease, a cancer, an ocular disease, a vascular disease, and a thyroid disease.

27. A method for generating the modified pluripotent cell of any one of claims 1-19, comprising increasing the expression of CD16, CD32, or CD64 in said parental non-modified version of said pluripotent cell.

28. The method of claim 27, wherein said modified cell has a human, monkey, cow, pig, chicken, turkey, horse, sheep, goat, donkey, mule, duck, goose, buffalo, camel, yak, llama, alpaca, mouse, rat, dog, cat, hamster, or guinea pig origin.

29. The method of claim 27, wherein said modified pluripotent cell is derived from a HIP cell.

30. The method of claim 27, wherein said modified pluripotent cell is derived from a HIPO-cell.

31. The method of claim 27, wherein said modified pluripotent cell is derived from an iPSC.

32. The method of claim 27, wherein said modified pluripotent cell is derived from an ESC.

33. The method of claim 27, wherein said increased CD16, CD32, or CD64 expression results from introducing at least one copy of a human CD16, CD32, or CD64 gene under the control of a promoter into said parental version of said modified pluripotent cell.

34. The method of claim 33, wherein said promoter is a constitutive promoter.

35. A pharmaceutical composition for treating a disease, comprising a cell derived from the modified pluripotent cell of any one of claims 1-19 and a pharmaceutically-acceptable carrier.

36. The pharmaceutical composition of claim 35, wherein said derivative cell is selected from the group consisting of a chimeric antigen receptor (CAR) cell, an endothelial cell, a dopaminergic neuron, a pancreatic islet cell, a cardiomyocyte, a retinal pigment endothelium cell and a thyroid cell.

37. The pharmaceutical composition of claim 35, wherein said disease is selected from the group consisting of Type I Diabetes, a cardiac disease, a neurological disease, a cancer, an ocular disease, a vascular disease, and a thyroid disease.

38. A medicament for treating a disease, comprising a cell derived from the modified pluripotent cell of any one of claims 1-19.

39. The medicament of claim 38, wherein said derivative cell is selected from the group consisting of a chimeric antigen receptor (CAR) cell, an endothelial cell, a dopaminergic neuron, a pancreatic islet cell, a cardiomyocyte, a retinal pigment endothelium cell and a thyroid cell.

40. The medicament of claim 38, wherein said disease is selected from the group consisting of Type I Diabetes, a cardiac disease, a neurological disease, a cancer, an ocular disease, a vascular disease, and a thyroid disease.

41. A modified cell, comprising an elevated level of CD16, CD32, or CD64 protein expression when compared to a parental version of said modified cell, wherein said elevated protein expression causes said modified cell to be less susceptible to antibody dependent cellular cytoxicity (ADCC) or complement-dependent cytotoxicity (CDC).

42. The modified cell of claim 41, wherein said cell is selected from the group consisting of a chimeric antigen receptor (CAR) cell, an endothelial cell, a dopaminergic neuron, a pancreatic islet cell, a cardiomyocyte, a retinal pigment endothelium cell and a thyroid cell.
